(19) United States
(12) Patent Application Publication
(10) Pub. No.: US 2014/0050670 A1
(43) Pub. Date: Feb. 20, 2014

(54) MULTIFUNCTIONAL NANOPARTICLES

(71) Applicants: Killugudi L. Iyer, Crawley (AU); Cameron William Evans, Waikiki (AU); Tristan Devere Clemons, Ferndale (AU); Melinda Fitzgerald, Salter Point (AU); Sarah Alison Dunlop, Cleremont (AU); Igor Luzinov, Central, SC (US); Bogdan Zdyrko, Clemson, SC (US)

(72) Inventors: Killugudi L. Iyer, Crawley (AU); Cameron William Evans, Waikiki (AU); Tristan Devere Clemons, Ferndale (AU); Melinda Fitzgerald, Salter Point (AU); Sarah Alison Dunlop, Cleremont (AU); Igor Luzinov, Central, SC (US); Bogdan Zdyrko, Clemson, SC (US)

(73) Assignee: THE UNIVERSITY OF WESTERN AUSTRALIA, Nedlands, WA (AU)

(21) Appl. No.: 13/992,508

(22) PCT Filed: Dec. 7, 2011
(86) PCT No.: PCT/AU2011/001588

§ 371 Date: Oct. 25, 2013

(30) Foreign Application Priority Data
Dec. 7, 2010 (AU) 2010905384

(51) Int. Cl. A61K 49/08; C12N 15/113

(52) U.S. Cl. CPC A61K 49/085; C12N 15/113
USPC 424/9.322; 435/375; 435/455

(57) ABSTRACT

The invention provides a nanoparticle for delivery of therapeutic agents, comprising a polymeric nanosphere and one or more detection agents, said detection agents for use in detecting the location of the nanoparticle. The invention further provides a transfection agent comprising an aforementioned nanoparticle.

### FIELD OF THE INVENTION

The present invention relates to the field of polymeric nanoparticles. More particularly, the present invention relates to polymeric nanoparticles with properties for multimodal imaging and adapted for the delivery of diagnostic and therapeutic agents, and their use as transfection agents.

### BACKGROUND ART

Nanoparticle technology is a rapidly emerging field and has advanced to clinical applications in the last few years. High expectations have been raised for the development of novel high-resolution diagnostics and drug nanocarriers for more efficacious and personalized therapies.

Drug delivery focuses on maximizing bioavailability both at specific places in the body and over a period of time. In this respect, the use of nanoparticles in drug delivery and for delivery of other therapeutic agents holds great promise as a viable means for preventing and treating a variety of diseases through delivery of drugs and other agents with targeted precision and adjustable release times.

Current multifunctional nanoparticles generally consist of a non-degradative, biocompatible matrix such as mesoporous silica, or a polymer in which the payload is encapsulated. However, for applications including drug delivery: controlled delivery to a particular tissue, drug stability, target accessibility, tissue permeability and penetrability, immunogenicity, and toxicity from the exclusive accumulation of nanoparticles in a targeted issue, are current major challenges limiting their broad clinical application. This is particularly important when cytotoxic substances are to be delivered by nanoparticles.

The preceding discussion of the background art is intended to facilitate an understanding of the present invention only. The discussion is not an acknowledgement or admission that any of the material referred to is or was part of the common general knowledge as at the priority date of the application.

### SUMMARY OF THE INVENTION

The importance of understanding endocytotic mechanisms has been previously overlooked in multifunctional nanoparticle design, particularly where intracellular delivery or sensing is desired. Thus, through gaining greater understanding of these mechanisms, the inventors have produced the nanoparticle of the invention which through its design is multifunctional, having advantages in multimodal imaging, delivery of agents and in its incorporation into cells and tissue whilst reducing issues of toxicity.

The invention provides a nanoparticle comprising a polymeric nanosphere and one or more detection agents, said detection agents for use in detecting the location of the nanoparticle.

In a preferred form of the invention, the polymer of the polymeric nanosphere comprises epoxide functional groups. In a preferred embodiment, the polymer is poly(glycidyl methacrylate) (PGMA).

The detection agent of the nanoparticle preferably comprises one or more surface modifying agents. The surface modifying agent may comprise a polycation. The polycation may be a polycation selected from: poly(ketimines), poly(amino acids), poly(guanidiriium), poly(alkylamines), poly(arylamines), poly(alkenylamines), and poly(alkynylamines), such as poly(imidazoles), poly(pyridines), poly(pyrimidines), poly(pyrazoles), poly(lysine), branched or linear poly(ethyleneimine), poly(histidine), poly(ornithine), poly(arginine), poly(asparginine), poly(glutamine), poly(tryptophan), poly(vinylpyridine), cationic guar gum, or copolymers, or mixtures thereof. Other, preferred surface modifying agents may comprise polyethylenimine (PEI), poly(ethylene glycol) (PEG), and/or PEG copolymers.

An additional or alternative detection agent of the nanoparticle of the invention may comprise one or more imaging labels. These include imaging labels which can be detected using the following imaging techniques: Fluorescence and magnetic resonance imaging (MRI); Fluorescence and positron emission tomography (PET); Fluorescence and x-ray computed tomography (CT); Fluorescence and MRI and PET and CT. A preferred imaging label is iron oxide nanoparticles. Another preferred imaging label is a fluorescent dye, a non-limiting example of which is Rhodamine B (RhB).

In a preferred form of the invention, the nanoparticle comprises a therapeutic agent. The nanoparticle may comprise one or more therapeutic agent selected from the group comprising: DNA, RNA, polypeptide, antibody, antigen, carbohydrate, protein, peptide, enzyme, amino acid, hormone, steroid, vitamin, drug including a slow release drug, virus, polysaccharides, lipids, lipopolysaccharides, glycoproteins, lipoproteins, nucleoproteins, oligonucleotides, immunoglobulins, albumin, haemoglobin, coagulation factors, peptide and protein hormones, non-peptide hormones, interleukins, interferons, cytokines, peptides comprising a tumour-specific epitope, cells, cell-surface molecules, small organic molecules, small organometallic molecules, nucleic acids and oligonucleotides, metabolites of or antibodies to any of the above substances.

The invention further provides a transfection agent for transfecting a cell with nucleic acid, comprising a nanoparticle of the invention as described herein. The nucleic acid that may be transfected may be one or more from the group comprising: plasmid DNA, vector DNA, siRNA, shRNA, genomic DNA, small or synthetic oligonucleotides nucleic acids and oligonucleotides comprise genes, viral RNA and DNA, bacterial DNA, fungal DNA, mammalian DNA, cDNA, mRNA, miRNA, miRNA mimics, miRNA inhibitors, piRNA, RNA and DNA fragments, modified oligonucleotides, single stranded and double-stranded nucleic acids, natural and synthetic nucleic acids. In a preferred form of the invention, the ratio of the nanoparticle to nucleic acid in the transfection agent is approximately 1:20. In another form, the ratio of the nanoparticle to nucleic acid in the transfection agent is approximately 1:4.

The invention provides a use of a transfection agent comprising a nanoparticle of the invention as described herein, for modulating phenotypic changes in a cell, tissue or a subject. The transfection agent may comprise a miRNA mimic to increase the function of endogenous miRNA to assist detection of a phenotypic change in a cell, tissue or a subject; or a miRNA inhibitor to decrease or eliminate the function of endogenous miRNA, increase expression of a target gene, and thereby modulate a change to a phenotype in a cell, tissue or subject.

The invention also provides a use of a transfection agent comprising a nanoparticle of the invention as described herein, for modulating the expression of a gene in a cell, tissue or a subject. Modulating the expression of the gene includes increasing, decreasing, or eliminating levels of polypeptide product of the gene in the cell, tissue, or a subject, as compared to normal levels of the polypeptide within the cell, tissue, or subject. A transfection agent comprising a nanoparticle of the invention as described herein may be used to decrease or eliminate expression of the gene which in turn decreases or eliminates levels of polypeptide product of the gene in tumour or cancer cells, as compared to normal levels of the polypeptide within the tumour or cancer cells. Alternatively, a transfection agent comprising a nanoparticle of the invention as described herein may be used to cross the blood brain barrier of the subject to modulate gene expression in the central nervous system.

The invention provides for a use of a nanoparticle of the invention as described herein, in the preparation of a medicament for the treatment of a disease associated with undesirable levels of a polypeptide in the cells, or tissue of a subject.

The invention also provides for a use of a transfection agent comprising a nanoparticle of the invention as described herein, in the preparation of a medicament for the treatment of a disease associated with undesirable levels of a polypeptide in the cells, or tissue of a subject. In one example, the transfection agent comprising a nanoparticle of the invention as described herein is used in the preparation of a medicament for the treatment of cancer or tumours.

The cells that can be transfected using the transfection agent may be a variety of different cell types in a subject, in a culture or individual cells.

The invention also provides a method for transfecting a cell comprising use of a transfection agent comprising a nanoparticle of the invention as described herein.

In a further embodiment, the invention provides a method for modulating the expression of a gene in a cell, tissue or a subject, the method including the step of introducing to the cell, tissue or subject a transfection agent comprising a nanoparticle of the invention as described herein. In this method, modulating the expression of the gene may increase, decrease, or eliminate levels of polypeptide product of the gene in the cell, tissue, or a subject, as compared to normal levels of the polypeptide within the cell, tissue, or subject. In one example, the invention provides a method for decreasing or eliminating expression of a gene in a cell, comprising transfecting the cell with a transfection agent comprising a nanoparticle of the invention as described herein, wherein the transfection agent comprises shRNA or siRNA complementary to the gene that can decrease or eliminate (otherwise known as ‘silence’) the expression of a protein product from the gene.

The present invention further provides a method for preparing and producing a transfection agent comprising a nanoparticle of the invention as described herein.

### BRIEF DESCRIPTION OF THE FIGURES

FIG. 1. Fluorescent, superparamagnetic nanospheres were prepared by an emulsion route and made use of the reactive epoxide groups of PGMA to anchor PEI. (a) Schematic representation of the attachment of PEI to fluorescent PGMA RhB nanospheres containing iron oxide nanoparticles. (b) Iron oxide (magnetite, Fe₃O₄) nanoparticles prepared by high temperature decomposition (scale bar=100 nm). (c) Low-magnification image of polymeric nanospheres (scale bar=500 nm). (d) Individual polymer nanosphere showing distribution of iron oxide nanoparticles within the polymer shell (scale bar=20 nm). (e) Particle size (left panel) and zeta potential (right panel) distributions before (open) and after (solid) modification with PEI.

FIG. 2. (a) The maximum intensity projection of nanospheres in PC12 cells; (b) the uptake of nanospheres in a PC12 culture as quantified by fluorescence over time; (c) confocal images of nanosphere uptake by PC12 cells with panels (from top to bottom) showing DIC, fluorescence and overlay images, recorded at times (from left to right) 0, 0.5, 1, 2, 3, 6, 12, 18, 24 hr (scale bar 20 μm);

FIG. 3. PEI-modified polymeric nanospheres (10 μg mL⁻¹) are rapidly internalized. (a) Confocal maximum intensity projection of nanospheres in PC12 cells after 72 hr (red (white in greyscale)=RhB, nanospheres; blue (grey in greyscale)=Hoechst, nuclei; DIC overlay; 40x/1.25, scale bar=20 μm). (b) Epifluorescence image of nanosphere uptake in rMC-1 cells after 24 hr (red (white in greyscale)=RhB, nanospheres; blue (grey in greyscale)=Hoechst, nuclei; 20x/0.50, scale bar=100 μm). (c) Hippocampal neuron as viewed by confocal microscopy after 24 hr incubation with nanospheres (red (white in greyscale)=RhB, nanospheres; green (dark grey in greyscale)=β-III-tubulin/Alexa 488, neurons; blue (light grey in greyscale)=Hoechst, nuclei; 60x/1.49, scale bar=5 μm). (d) Confocal image of a cortical neuron after 24 hr incubation with nanospheres (red (white in greyscale)=RhB, nanospheres; green (dark grey in greyscale)=β-III-tubulin/Alexa 488, neurons; blue (light grey in greyscale)=Hoechst, nuclei; 60x/1.49, scale bar=5 μm). (e) Frames from a region of confocal time-lapse experiment (0, 0.5, 1, 2, 3, 6, 9, 12, 18, 24 hr) showing internalization of nanospheres by PC12 cells (red (white in greyscale)=RhB, nanospheres; DIC overlay; 20x/0.75, scale bar=10 μm). (f) Nanosphere uptake in PC12 cells over time quantified by RhB fluorescence.

FIG. 4. Elemental mapping by EFTEM identifying iron within cellular samples, revealing the location of nanospheres;

FIG. 5. PEI-modified polymer nanospheres (10 μg mL⁻¹ in all preparations) are visualized by TEM at various time points displaying stages in internalization and compartmentalization. (a) Endocytosis of nanospheres by a macropinocytotic-like route (nanospheres indicated by arrows; scale bars=100 nm). (b) Endocytosis that is apparently clathrin—and caveolinindependent (nanospheres indicated by arrows, and internalized particles by arrowheads; scale bar=200 nm). (c) Time series showing stages in intracellular trafficking. From left to right, at 3 hr, nanospheres have been internalized; at 6 hr, they form loose clusters; at 12 hr, become associated with other membrane-bound vesicles; and at 24 hr, are located in clusters bound by multiple membranes (nanospheres are outlined as a visual aid; arrows indicate multiple membranes surrounding nanospheres; scale bars=100, 100, 200, 100 nm). (d) After 72 hr, nanospheres are arranged in discrete clusters, many of which are surrounded by multiple membranes (arrows indicate multiple membranes surrounding nanosphere clusters; arrowheads indicate membranes apparently in the process of fusion, expanded in the inset; scale bars=200 nm). (e) Whole cell section constructed from three images after 72 hr incubation with nanospheres. Nanospheres are visible at the cell surface (arrowhead) and in clusters (arrows) throughout the section (false-colored image; scale bar=1 μm).

FIG. 6. The linear fit for fluorescence as a function of iron content within the nanospheres, with standard samples of free nanospheres (open symbols) and nanospheres in a cellular sample (filled symbol).

FIG. 7. Fluorescent, superparamagnetic nanospheres were prepared by an emulsion route and made use of the reactive epoxide groups of PGMA to anchor PEI. (a) Schematic representation of the attachment of PEI to fluorescent PGMA RhB nanospheres containing iron oxide nanoparticles. (b) Iron oxide (magnetite, Fe₃O₄) nanoparticles prepared by high temperature decomposition (scale bar=100 nm). (c) Low-magnification image of polymeric nanospheres (scale bar=500 nm). (d) Individual polymer nanosphere showing distribution of iron oxide nanoparticles within the polymer shell (scale bar=20 nm). (e) Particle size (left panel) and zeta potential (right panel) distributions before (open) and after (solid) modification with PEI.

FIG. 8. PEI-modified polymeric nanospheres (10 μg mL⁻¹) are rapidly internalized. (a) Confocal maximum intensity projection of nanospheres in PC12 cells after 72 hr (red (white in greyscale)=RhB, nanospheres; blue (grey in greyscale)=Hoechst, nuclei; DIC overlay; 40x/1.25, scale bar=20 μm). (b) Epifluorescence image of nanosphere uptake in rMC-1 cells after 24 hr (red (white in greyscale)=RhB, nanospheres; blue (grey in greyscale)=Hoechst, nuclei; 20x/0.50, scale bar=100 μm). (c) Hippocampal neuron as viewed by confocal microscopy after 24 hr incubation with nanospheres (red (white in greyscale)=RhB, nanospheres; green (dark grey in greyscale)=β-III-tubulin/Alexa 488, neurons; blue (light grey in greyscale)=Hoechst, nuclei; 60x/1.49, scale bar=5 μm). (d) Confocal image of a cortical neuron after 24 hr incubation with nanospheres (red (white in greyscale)=RhB, nanospheres; green (dark grey in greyscale)=β-III-tubulin/Alexa 488, neurons; blue (light grey in greyscale)=Hoechst, nuclei; 60x/1.49, scale bar=5 μm). (e) Frames from a region of confocal time-lapse experiment (0, 0.5, 1, 2, 3, 6, 9, 12, 18, 24 hr) showing internalization of nanospheres by PC12 cells (red (white in greyscale)=RhB, nanospheres; DIC overlay; 20x/0.75, scale bar=10 μm). (f) Nanosphere uptake in PC12 cells over time quantified by RhB fluorescence.

FIG. 9. Relaxometry shows effects of compartmentalization of PEI-modified polymeric nanospheres in PC12 cells. In all panels, solid points represent free nanospheres and open points cell-bound nanospheres. (a) Decrease in r2 for cell-bound nanospheres compared with free nanospheres. (b) Decrease in r1 following internalization of nanospheres. (c) Effect of echo spacing (TE) on R2 relaxation rate. Top panel: Similar values of r2 for free nanospheres using short (triangles) and long (stars) TE. Bottom panel: Nanospheres compartmentalized within cells display a doubling in r2 between short (triangles) and long (stars) echo time measurements. (d) Reduced dependence of r1 on field for compartmentalized (open) compared with free (solid) particles.

FIG. 10. Lomerizine release from nanospheres with and without PEI modification (LNP±PEI) over time. Filled symbols show release at pH 5, and open symbols the release at pH 6. In both cases, no release of lomerizine was detectable by HPLC at pH 7.4 (not shown). a) Release from nanospheres without modification (LNP−PEI). b) Release from nanospheres with PEI modification (LNP+PEI). Error bars denote SE.

FIG. 11. Intracellular calcium concentration is reduced in cells treated with lomizine delivered using nanospheres following glutamate challenge. a) Both lomerizine delivered using nanospheres modified with PEI (LNP+PEI) and 1 μM free drug caused similar reductions in intracellular calcium [Ca²⁺]ᵢ after exposure to glutamate (10 mm, 24 hr). Lomerizine-loaded nanospheres without PEI modification (LNP−PEI) do not release lomerizine and therefore [Ca²⁺]ᵢ is not reduced after glutamate injury. All statistical significances relative to ENP (vehicle); *p≦0.05, **p≦0.01, ***p≦0.001; one-way ANOVA with Bonferroni post hoc correction. b) Representative traces showing differences in Fura-2 fluorescent ratios (R=F₃₄₀/F₃₈₀) recorded in single PC12 cells after nanosphere treatment with either LNP+PEI or ENP and 24 hr exposure to glutamate; ionomycin was added at 5 min, and Rₘₐₓ was determined from the subsequent [Ca²⁺]ᵢ rise.

FIG. 12. DNA binding and retardation assay. Fixed concentration of oligos and vector DNA with varying concentration of PEINPs were incubated with HEPES buffer pH 7.2 for 30 min at room temperature. The DNA-(PGMA-PEI-nanoparticle) (PEINP) were analysed on 0.8% agarose gel. (a) represents oligos, and (b) represents vector DNA retardation at 1:0.4 and 1:25 respectively as compared with DNA and PEINPS controls.

FIG. 13. Comparison of transfection efficiency of lipofectamine (a-f) with PEINPs (g-l) in adherent, semi-adherent and non-adherent cell lines. PEINPs-DNA (1:20) and lipofectamine-DNA complexes were used to transfect cell lines mentioned above. Rhodamine and the green fluorescent protein (GFP) expression was observed under fluorescent microscope at 63× magnification.

FIG. 14. Transfection efficiency of PEINPs was confirmed in HEK 293 cell lines using FACS. PEINPs with and without rhodamine were used to transfect HEK 293 cells and rhodamine linked to the cells as well as GFP expression were analysed by fluorescence-activated cell sorter (FACS) after 48 hrs. (a). represents histogram plot of control cells. (b), (c) & (d) represent density plots of cells transfected with PEINP with and without rhodamine respectively.

FIG. 15. Analysis of target gene knockdown and recovery in different cell lines (a. HEK293, b. MDA-MB231 and c. Jurkat) after PEINP-microRNA transfections. The transfected cells were processed for immunocytochemistry, q-per and western analysis after 48 hr. Beta-actin was used as loading control. Anti-rabbit-alexa 488 was used and images were taken at 63× using confocal microscopy.

FIG. 16. Analysis of housekeeping gene (GAPDH) knockdown in different cell lines (a. HEK293, b. MDA-MB231 and c. Jurkat) after PEINP-shRNA transfections. The transfected cells were processed for immunocytochemistry, q-per and western analysis after 48 hr. Beta-actin was used as loading control. Anti-rabbit-alexa 633 was used and images were taken at 63× using confocal microscopy.

FIG. 17. Analysis of oncogene gene (C-myc) knockdown in different cell lines (a. HEK293, b. MDA-MB231 and c. Jurkat) after PEINP-shRNA transfections. The transfected cells were processed for immunocytochemistry, q-per and western analysis after 48 hr. Beta-actin was used as loading control. Anti-rabbit-alexa 488 was used and images were taken at 63× using confocal microscopy.

FIG. 18. Imaging of mouse injected with PEINP & PEINP-c-myc-shRNA. (a). Multispectral imaging (merged with X-ray) shows containment of rhodamine in the injected tumours and not in any other organs. (b). In vivo axial and sagittal imaging of tumours after 48 hr of injection with PEINPs (red circle indicates penetration of PEINPS to tumour necrotic core). (c). Confocal imaging showing the biodistribution of PEINPs throughout tumour core and periphery.

FIG. 19. Tumour regression and survival of breast cancer mice models: PEINPS-C-Myc shRNA complex injection in the tumour resulted significant regression in tumour size and prolonged survival as compared with untreated mice models. The C-Myc transcript and protein level were quantified by Q-PCR and immunohistochemistry respectively where quite significant low level of C-Myc when compared with untreated tumour mice model.

FIG. 20. Oral delivery and retention of PEINPs in colon cancer mice models. Confocal microscopy was performed in order to locate the PEINPs and PEINPs+DNA (GFP) in the gut of colon cancer mice model after 48 hr of oral delivery. Following that, 95% accumulation of PEINPs was observed in the colon. GFP expression was also observed as shown in the DNA-GFP panel of the figure.

FIG. 21. Embryofection of linear and plasmid DNA stained with Hoechst was performed using PEINPs in mouse embryos. These DNAs stained with Hoechst satin complexed with PEINPs were incubated with mouse embryo at single cell stage till embryo grows up to four cell stage ex vivo and were observed under confocal microscopy. These experiment shows that PEINPs could efficiently bind to embryos delivered DNAs as evident with the Hoechst and GFP panels compared with un-incubated embryos. These Images were taken at 10× and were reviewed at 3× to confirm the PEINPs and DNA localization inside the embryos.

### DETAILED DESCRIPTION OF PREFERRED EMBODIMENTS OF THE INVENTION

### General

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (e.g. size, concentration etc). A range of values will be understood to include all values within the range, including the values defining the range, and values adjacent to the range which lead to the same or substantially the same outcome as the values immediately adjacent to that value which defines the boundary to the range.

Throughout this specification, unless the context requires otherwise, the word “comprise” or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Reference to information contained in the text should not be understood as a concession that the material or information was part of the common general knowledge or was known in Australia or any other country.

Features of the invention will now be discussed with reference to the following non-limiting description and examples.

In a first aspect of the invention, the invention comprises a nanoparticle comprising a polymeric nanosphere and one or more detection agents, wherein said one or more detection agents can be used for detecting the location of the nanoparticle. In a preferred embodiment, the polymeric nanosphere comprises a polymer containing epoxide functional groups. In a more-preferred embodiment, the polymer is poly(glycidyl methacrylate) (PGMA).

A polymer comprising multiple epoxy groups and having a molecular weight of at least about 2000 can be used to prepare the nanoparticles of the invention. In addition, the polymer can be cross-linked to form a cross-linked polymeric nanoparticle that contains additional epoxy functionality throughout the particle. In one embodiment, the polymer can be cross-linked to the epoxy groups on the polymer. For instance, between about 10% and about 40% of the epoxy groups on the polymer can be utilized to cross-link the polymer. Preferably, the polymer used to prepare nanospheres has M_{w} 200-300 kDa (nominally 250 kDa).

Preferably the diameter of the nanosphere is between 90 nm and 260 nm with a Z-average diameter of around 170 nm.

The nanoparticle of the invention comprises one or more detection agents of the same type. The nanoparticle may also comprise one or more different detection agents. In one embodiment of the invention one or more detection agents are surface modifying agents.

The surface properties of nanoparticles play an important role in determining the outcome of their interactions with cells for developing effective therapies. Epoxy groups present on the particle surface can be highly reactive under a wide variety of conditions. For instance, epoxy can react with any of carboxy, hydroxy, amino, thiol, or anhydride functional groups under a variety of conditions. As such, the epoxy-containing polymers of the present invention can be readily bound to other surfaces via available functionalities. The surface of the nanoparticles of the invention serves as a universal platform for anchoring, for example, amongst others, therapeutic agents, targeting ligands or payloads such as antibodies, enzymes, peptides, nucleic acids, folic acid, specific polymers for tailoring cellular uptake, and specific molecules to introduce particular functional groups.

In one embodiment, the nanosphere surface modifying agent is polyethylenimine (PEI). PEI can alter the surface properties of the polymeric nanosphere to induce a positive charge. In a second embodiment, the nanosphere surface modifying agent is PEG. PEG can alter the surface properties of the polymeric nanosphere such that circulation times of the nanoparticle, for example, in a living organism such as a human, may be prolonged in vivo.

The one or more detection agents of the nanoparticle of the invention may comprise one or more imaging labels. Said imaging labels may be recognised using imaging techniques. Said imaging techniques may comprise medical imaging techniques which include radiography and investigative radiological sciences, nuclear medicine, endoscopy, medical thermography, medical photography, microscopy, and more specifically, as some non-limiting examples, magnetic resonance imaging (MRI), electron microscopy, fluorescence microscopy, positron emission tomography (PET), X-ray tomography, luminescence (optical imaging), ultrasound, and magnetoencephalography (MEG), amongst others.

It is often necessary to use more than one imaging technique to integrate the strengths of each whilst overcoming the limitations of the individual techniques to improve diagnostics, preclinical research and therapeutic monitoring. However, each of these techniques typically require the administration of different agents to enable tracing or to improve contrast, so using more than one imaging technique requires additional time, expense and can complicate the diagnostic process. Thus, the nanoparticle of the invention, as described herein, has been constructed such that it may comprise one or more imaging labels. These imaging labels enable imaging techniques to be used to determine the location of the nanoparticle in, for example, a cell, biological tissue, or in a living organism. Such imaging techniques will be selected according to an imaging label incorporated into the nanoparticle of the invention. The benefit of multiple imaging labels is that better resolution of images is provided in addition to data on different length scales by exploiting the advantages offered by the individual techniques.

Imaging labels which may be incorporated into the nanoparticle of the invention may include magnetic species, radionuclides or radiolabelled compounds, radioactive nanoparticles, proteins, antibodies, antigens, fluorescent dyes, quantum dots, or therapeutic agents, amongst others.

In one embodiment, the imaging label is an iron oxide, an example of which is magnetite (Fe₃O₄), and in particular, nanoparticles of iron oxide. Iron oxide may be used as a negative contrast agent in T₂-weighted images for MRI. Thus, when a nanoparticle of the invention comprises an imaging label of an iron oxide, MRI may be used to determine or track the location of the nanoparticle in vivo (i.e. whilst inside, for example, a living organism).

Iron oxide nanoparticles for use as an imaging label in the nanoparticle of the invention may be produced by the high temperature decomposition of Fe(acac)₃ in the presence of oleic acid, oleyl amine, and 1,2-tetradecanediol or 1,2-hexadecanediol.

The incorporation of multiple iron oxide nanoparticles into the nanoparticle of the invention may be beneficial in that it can increase relaxation properties of the nanoparticles (R₂) providing higher contrast and better sensitivity for medical imaging using MRI. A preferred size of iron oxide nanoparticle for the purposes of the invention is around 4-12 nm in diameter.

In another embodiment, the imaging label is Rhodamine B (RhB). RhB is a fluorescent dye that contains a reactive carboxylic acid group, an important structural feature that enables the dye to be bound to the polymer nanoparticle of the invention.

RhB may be detected using techniques including fluorescence microscopy, confocal microscopy, flow cytometry, fluorescence correlation spectroscopy and enzyme-linked immunosorbent assay (ELISA). RhB is covalently bound to the polymer nanoparticle of the invention.

In a further embodiment of the invention, the nanoparticle described herein comprises the nanosphere surface modifying agents PEI and PEG and the imaging labels RhB and iron oxide nanoparticles. Such nanoparticles are useful in biological sensing and separation, particularly when these nanoparticles are modified by attaching binding proteins such as antibodies which achieves even greater target specificity.

The present invention further comprises a nanoparticle described herein adapted to deliver an agent. Said agent may comprise a variety of therapeutic or diagnostic agents. Therapeutic agents may include drugs and thus, the nanoparticle of the invention may be used as a drug delivery system. This may comprise a slow-release drug delivery system or the nanoparticle may be adapted to deliver a drug to a specific targeted site within a cell, biological tissue or a living organism. Thus, the nanoparticle of the invention may be used in the therapeutic treatment of diseases in humans and animals. This may be particularly beneficial when therapeutic treatment with a drug requires large regular doses in order to produce the therapeutic effect. Using a nanoparticle of the invention can provide extended, controlled release of the drug at the targeted treatment site.

Other agents that may be delivered by the nanoparticle of the invention comprise, in some non-limiting examples, small-molecule drugs, DNA, oligonucleotides, siRNA and/or nanoparticles for thermal treatment of tumours.

The polymeric nature of the nanoparticles enables the entrapment of therapeutic agents and drugs. Drugs are included in the mixture used to make the nanospheres and as the polymer precipitates during the emulsification, the drug becomes trapped inside the loosely aggregated polymer chains. Drugs are not permanently bound to the structure, however, and may be subsequently released by diffusion of the entrapped drug from the nanosphere into the surrounding medium.

The present invention further provides a nanoparticle described herein comprising surface modifications by surface modifying agents, one or more labels that can be recognised using imaging techniques and is adapted to deliver an agent, thus, providing polymeric nanoparticles that can be used for simultaneous imaging and therapy.

In one form of the invention, the nanoparticle described herein comprises the nanosphere surface modifying agents PEI and/or PEG and the imaging labels RhB and iron oxide nanoparticles, and is adapted to deliver a therapeutic agent. Preferably, the therapeutic agent is a drug.

The present invention further provides a method for producing a nanoparticle described herein. The method involves the aqueous emulsification of an organic phase containing non-water soluble ingredients in the presence of an emulsifier or surfactant. Preferably, the method is a modified non-spontaneous nanoprecipitation, in which a solvent or mixture of solvents is used as the organic phase.

The present invention further provides a pharmaceutical composition comprising the nanoparticle described herein.

The present invention also relates to medicaments produced using said nanoparticles described herein and to methods of treatment of an animal, including a human, using a therapeutically effective amount of said nanoparticles described herein administered by way of said medicaments.

A nanoparticle of the invention as described herein may be combined with one or more pharmaceutically acceptable carriers, as well as any desired excipients or other like agents commonly used in the preparation of medicaments.

The invention further provides a transfection agent comprising a nanoparticle of the invention as described herein, preferably with high transfection efficiency with no or minimal cytotoxicity of the nanoparticles to the cell in which introduced. In this respect, said transfection agent comprising a nanoparticle of the invention can be used in the delivery of biomolecules to a cell. More specifically, said transfection agent comprising a nanoparticle of the invention can deliver various nucleic acids, such as plasmid DNA, vector DNA, siRNA, shRNA, genomic DNA, small or synthetic oligonucleotides nucleic acids and oligonucleotides comprise genes, viral RNA and DNA, bacterial DNA, fungal DNA, mammalian DNA, cDNA, mRNA, miRNA, miRNA mimics, miRNA inhibitors, piRNA, RNA and DNA fragments, modified oligonucleotides, single stranded and double-stranded nucleic acids, natural and synthetic nucleic acids, amongst others, into a cell.

In one non-limiting example, in a transfection agent comprising a nanoparticle of the invention, the ratio of nanoparticle to oligonucleotides is preferably between approximately 1:2 to 1:6. More preferably, the ratio of nanoparticle to oligonucleotides is approximately 1:4. In another example, in a transfection agent comprising a nanoparticle of the invention, the ratio of nanoparticle to nucleic acid is preferably between approximately 1:15 to 1:25. More preferably, the ratio of nanoparticle to nucleic acid is approximately 1:20.

The transfection agent comprising a nanoparticle of the invention may be employed in the delivery of the aforementioned nucleic acids to adherent cells, semi-adherent cells, non-adherent cells, tissue, or a subject, and particularly with transfection efficiency comparable to that of viral transduction.

Thus, the invention provides a method for modulating the expression of a gene in a cell, tissue or a subject, the method including the step of introducing to the cell, tissue or subject a transfection agent comprising a nanoparticle of the invention. Modulating the expression of a gene may include the down regulation or ‘silencing’ of a gene to reduce or prevent expression of a polypeptide product of the gene in a cell, tissue, or a subject, as compared to normal levels of the polypeptide within the cell, tissue, or subject. miRNA mimics may be used to increase the function of a particular endogenous miRNA for easier detection of a phenotypic change in a cell, tissue or a subject. Alternatively, miRNA inhibitors may be used to decrease or eliminate (suppress) the function of particular endogenous miRNAs, increase the expression of the target gene, and decrease or attenuate the presentation of a phenotype in a cell, tissue or a subject.

In one non-limiting example, the transfection agent comprising a nanoparticle of the invention may be directed to an appropriate site of action which may comprise delivery of nucleic acids with a therapeutic benefit or other to tumour cells or a solid tumour. Thus, the transfection agent comprising a nanoparticle of the invention may provide transport of nucleic acids to a specific location such as a cancer or tumour, or an organ such as the colon, in a subject thereby avoiding damage to other tissue or organs, release of the nucleic acid in the specified location with a controlled concentration-time delivery profile, and can stop further expression of a target gene efficiently.

Moreover, a transfection agent comprising a nanoparticle of the invention may also be used as a carrier of a nucleic molecule such as shRNA or miRNA for targeting silencing of functional gene. In one non-limiting example, a transfection agent comprising a nanoparticle of the invention and a nucleic acid such as a gene c-myc shRNA could be used to down regulate expression of the c-myc gene which is known in the art to have unregulated expression or overexpression in certain cancers. A benefit in using a transfection agent comprising a nanoparticle of the invention is the hyperthermia produced by the superparamagnetic core of the nanoparticle which can assist in increasing transfection efficacy and efficient nucleic acid delivery to the necrotic core of a tumour. In another example, transfection agents comprising nanoparticles of the invention may be delivered to embryos to create transgenic animals, for example, transgenic mice.

Alternatively, modulating the expression of a gene may include the increased expression of a gene to increase expression of a polypeptide product of the gene in a cell, tissue, or a subject, as compared to normal levels of the polypeptide within the cell, tissue, or subject. In one non-limiting example, this may include use of miRNA mimics and inhibitors into cells to induce or inhibit a specific phenotype. In this respect, miRNA mimics augment the function of endogenous miRNA for easier detection of a phenotypic change. Conversely, miRNA inhibitors suppress the function of endogenous miRNAs, increase the expression of the target gene, and attenuate the presentation of the phenotype. In another example, a transfection agent comprising the nanoparticle of the invention may be used to cross the blood brain barrier to modulate gene expression in the central nervous system.

The transfection agent comprising a nanoparticle of the invention may be labelled or ‘linked’ with a marker such as rhodamine, or another marker as described herein, for the purpose of visualising these nanoparticles, for example, using confocal imaging.

The invention further provides a process for preparing a transfection agent comprising the nanoparticle of the invention.

### EXAMPLES

### Example 1

### Preparation and Characterisation of Nanoparticles of the Invention

### Results and Discussion

### Preparation of Nanospheres by Covalent Reactions and Non-Spontaneous Emulsification

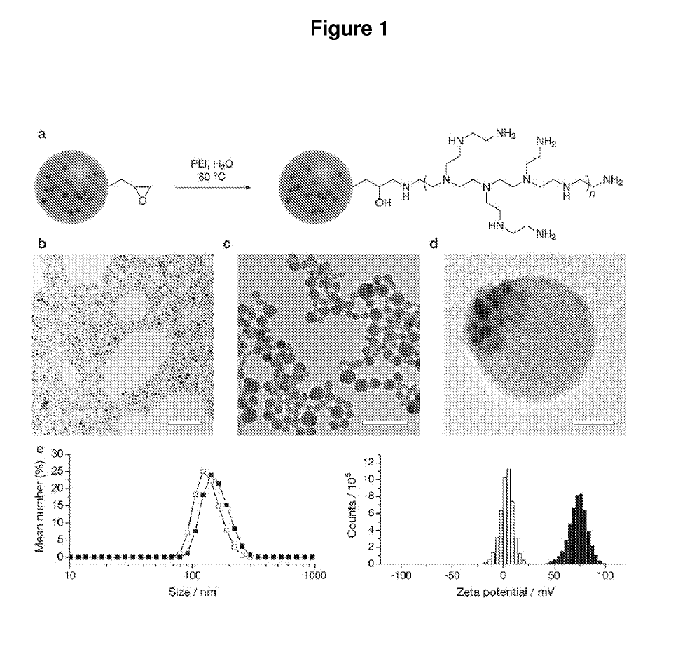

The preparation of magnetofluorescent polymer nanospheres used iron oxide nanoparticles synthesised by high temperature decomposition (FIG. 1b) and preformed polymers. Although electrostatic attraction may be employed in nanoparticle synthesis, such binding is reversible with the chance that imaging moieties may escape. Thus, it was preferable to attach surface modifying agents and dyes by covalent bonds. PGMA is a lipophilic polymer whose epoxide groups are reactive towards various nucleophiles, so covalent attachment to the polymer was possible by means of a simple ring-opening reaction. Because the majority of reactive epoxide groups would be inaccessible after forming nanospheres, RhB may be attached to the polymer in a solution-phase reaction. RhB is a xanthene dye that contains a reactive carboxylic acid group as described above, an important feature that enables the dye to be grafted to the polymer. Using the modified polymer, polymer nanoparticles were prepared (FIGS. 1c,d) using a modification of a nanoprecipitation technique, in which an organic solution of the dye-modified polymer and iron oxide nanoparticles was emulsified in water in the presence of a surfactant. The presence of iron oxide nanoparticles and modified polymer meant that spontaneous emulsification could not occur. Therefore, a non-miscible solvent with water (CHCl₃) in conjunction with a partially water-soluble solvent (MEK) were used which would aid in the emulsification process by diffusion of MEK into the aqueous phase and ensuring supersaturation of the organic phase.

Surface modifications were then made to the nanoparticles. In general, nanoparticles with positive surfaces are taken up non-specifically because cationic polymers interact with negatively charged species of the cell membrane and facilitate cellular uptake by fluid-phase or receptor-mediated endocytosis. Therefore, the nanospheres were then reacted with PEI in water, resulting in covalent attachment of PEI to the nanoparticle surface, again via the ring-opening of epoxide groups. Crosslinking of the polymer is expected to occur to some extent during the synthesis procedure but although it is possible to hydrolyse remaining epoxide groups, it was not carried out. The modified polymer nanospheres were collected by passing them through a magnetic separation column filled with coated iron beads in order to remove excess polymers and any non-magnetic nanospheres.

### Characterising the Polymer Nanospheres

The prepared polymer nanospheres were characterised using a range of techniques including dynamic light scattering (DLS), transmission electron microscopy (TEM), fluorescence spectrophotometry and magnetometry. The size, surface charge, and magnetic and fluorescence properties of the nanospheres was determined, as size and surface properties strongly influence biological responses to nanoparticles. The results show the nature of the particles that were prepared and their potential for use as multimodal imaging tools.

DLS is a technique that measures the hydrodynamic size of the nanospheres. Analysis before and after surface modification confirmed the attachment of PEI to the nanosphere surface; there was a small increase in average size and a large positive shift in the zeta potential distribution (FIG. 1e). 95% of the PEI-modified nanospheres had diameters between 90 and 260 nm, with a Z-average of 171 nm. TEM confirmed the DLS result. Elemental mapping by energy-filtered TEM (EFTEM) showed that there was a multitude of iron oxide nanoparticles contained within each polymer nanosphere and the general trend was for the iron oxide particles to be clumped on one side of the sphere. For the purposes of MRI, such an arrangement causes a considerable increase in R₂ relaxation rate of the particle assembly, improving the sensitivity of the probe. The aggregation of the iron oxide nanoparticles is presumably due to weak interaction between the lipophilic surface of the iron oxide and the polymer, although the solvent mixture employed in the emulsion process also dictated how well the iron oxide nanoparticles were dispersed in the nanospheres.

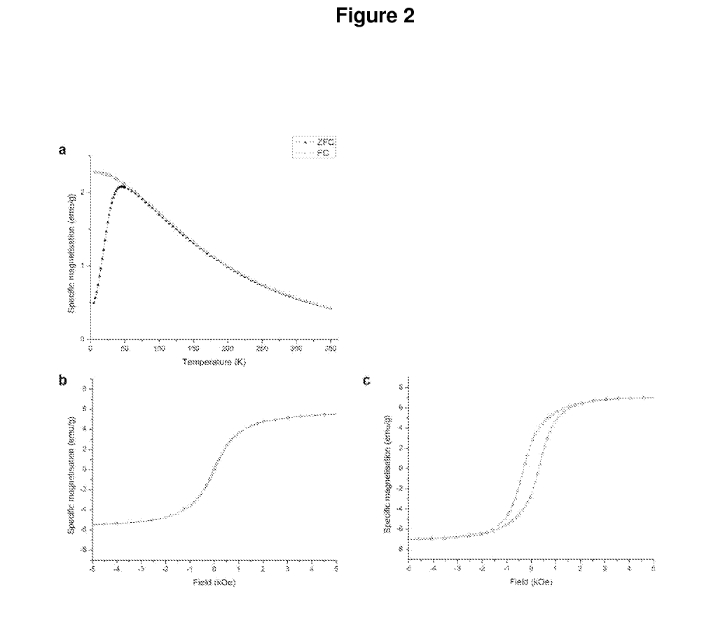

Magnetic measurements using a superconducting quantum interference device (SQUID) showed that the nanospheres retained the superparamagnetic property of the iron oxide nanoparticles and had a specific saturation magnetisation of 5.7 emu g⁻¹ (FIG. 2). The fluorescence excitation and emission spectra of the nanoparticles were essentially unchanged from that of RhB, and showed a maximum emission at approximately 590 nm. As the maximum emission wavelength of RhB is dependent on pH, the samples were buffered at pH 7.4.

It was found that the particle size distribution was sensitive to the molecular weight of the polymer used in the preparation, where either an increase or decrease in M_{w} caused significant broadening of the particle size distribution and an increase in average size.

The nanospheres contained both iron oxide nanoparticles and an organic dye and were therefore fluorescent and magnetic, and thus, it was expected that they could be visualised by TEM, optical microscopy, and MRI. Furthermore, they exhibited a relatively narrow size distribution and had a positively-charged surface due to attachment of PEI.

### Rapid Internalisation of Nanospheres by Cells

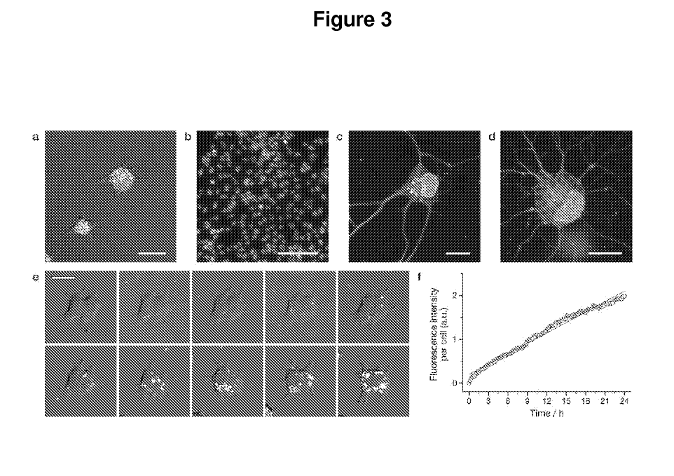

The PGMA nanospheres were incubated with rat pheochromocytoma (PC12) cells for a period of up to 72 hr. The rationalisation for selecting 72 hr was that endolysosomal escape of quantum dots was reported to occur after more than 24 hr. Particles with an aminated surface (i.e. modified with PEI) were taken up rapidly, even within the first few minutes, while those without the PEI modification were not associated with cells after 3 days. Particles that were internalised presented a punctuate distribution throughout the cells, but were excluded from the nucleus (FIG. 3).

PEI is known to act as a transfection agent and also particles with a positive surface charge are known to be internalised by cells more readily than negatively charged particles, which tend to adhere to cell membranes instead. However, PEI has been shown to have toxic effects in vitro, so the next step was to assess the toxicity of the particles using a variety of neuronal and non-neuronal cell types.

### Nanospheres do not Show Toxicity in Cell Culture Models

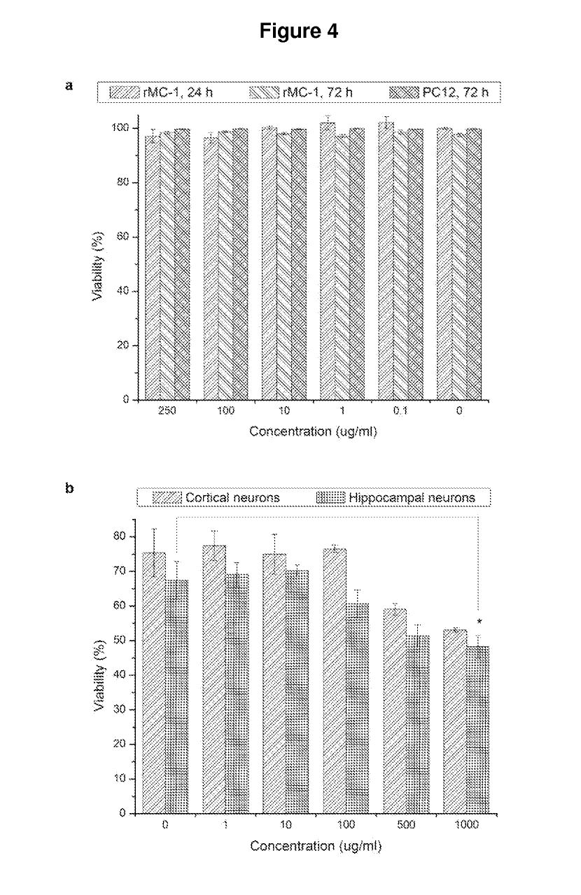

The toxicity of the polymer nanospheres was examined in rat pheochromocytoma (PC12), retinal Müller (rMC-1), rat hippocampal and rat cortical neuron cultures following 24-72 hr incubation using viability and live cell counts as outcome measures. For the immortalised cultures, cells were plated 24 hr prior to addition of nanospheres and were maintained in complete media throughout the experiment. Measurements conducted using Live/Dead cell viability kit revealed no statistically significant variations in viability (FIG. 4a), and neither live nor dead cell counts of immortalised cultures across the tested concentrations (p>0.05). Similar results were observed in the primary neuron cultures (FIG. 4b), although there was some evidence of a toxic effect at very high particle concentrations (≧0.5 mg ml⁻¹). This was not unexpected as the particle concentration was so high as to completely hide the cells from view. Trypan blue assay for PC12 cultures confirmed that there was no decrease in live cell numbers after incubation for either 24 hr or 72 hr. It was also confirmed that non-adherent dead cells were not being discarded in these experiments, which could falsely elevate the viability results.

While free PEI may compromise plasma membranes with a toxic effect, removing excess, unbound PEI here was shown to alleviate toxicity. As shown below, the nanospheres appeared not to escape from endolysosomal vesicles, which suggested that they do not permeabilise membranes, and are subsequently unable to interact with mitochondria. A possible reason as to why toxic effects were not observed with these nanospheres is because the magnetic nanospheres were washed on the column, thereby removing any excess PEI. Alternatively, the short chain length, or the relatively large particle size (compared to DNA/PEI transfection constructs, for example) may limit the effects of interaction between PEI and cell membranes.

### Uptake of Nanospheres can be Visualised by TEM

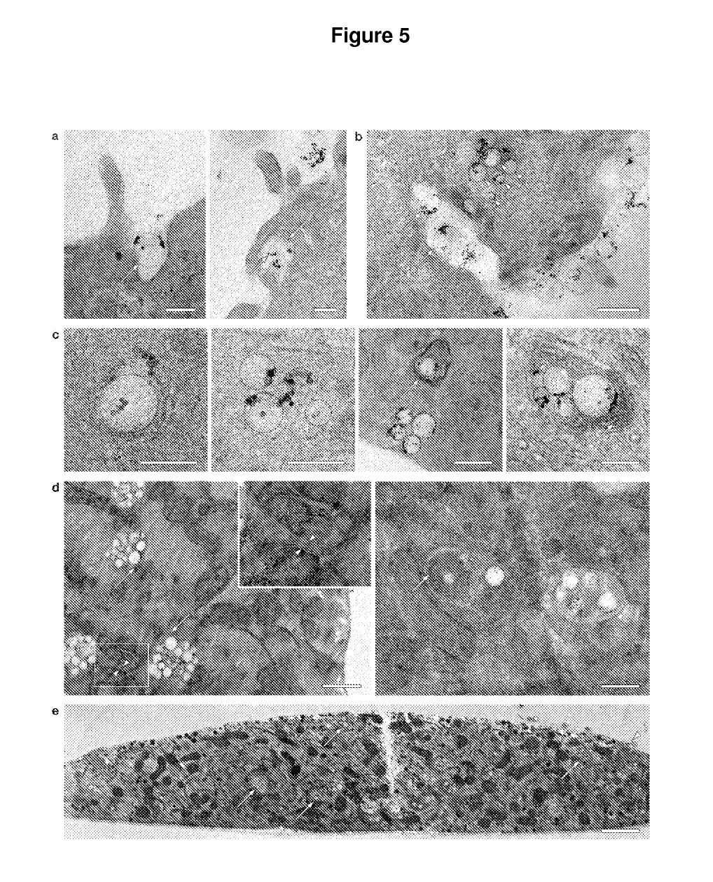

In order to better examine the nanosphere uptake and intracellular fate, PC12 cells were analysed by TEM. Cells were incubated with nanospheres (10 μg ml⁻¹) as above, but were grown on poly(L-lysine) and collagen-coated Aclar substrates to facilitate sample preparation, and were fixed after 3, 6, 12, 24 and 72 hr incubation. Inspection of the images (FIG. 5) furnished the following observations.

First, active endocytosis appeared to be the main route of particle entry into cells. In particular, macropinocytosis and uptake that was independent of clathrin and caveolae seemed to be taking place. Although particle uptake seemed to occur through these so-called non-receptor-mediated routes, particles were first associated with the cell surface, and then internalised after membrane invagination. Second, the nanospheres were mostly contained within membranes inside cells, even when there was only a single nanosphere visible, and some nanospheres were surrounded by multiple membranes. As time progressed, multiple vesicles combined; that is to say, the level of clustering of nanospheres within the cells increased. This suggests that the rate of endolysosomal escape is low, and that particles have little toxic effect through membrane disruption. Third, there appeared to be no free iron oxide nanoparticles in the cells, so either iron oxide nanoparticles were bound to the polymer or its lipophilicity held it in the particles, even through all the solvent-based TEM sample preparation steps.

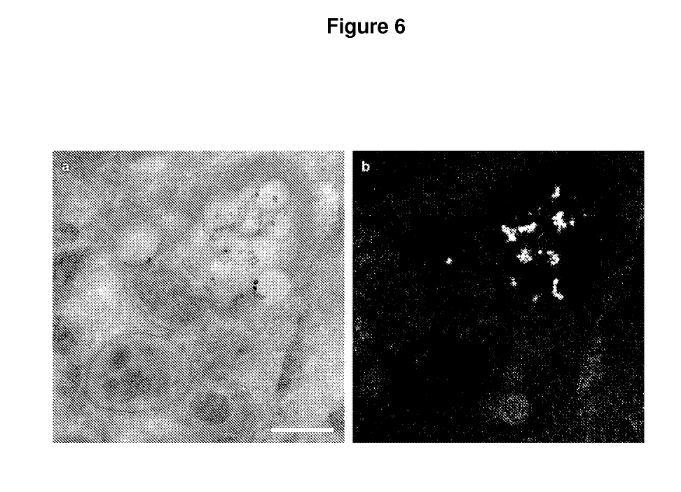

Just as EFTEM was used to demonstrate the presence of iron within nanospheres, it was used to identify iron within fixed tissue samples, revealing the location of nanospheres within cells (FIG. 6). Although the relatively high electron density of iron made its identification by TEM straightforward, the applicant was able to confirm that iron was present, and that there was no indication that any iron has escaped from the nanospheres.

### Nanospheres do not Appear to be in Lysosomes or Late Endosomes

Using the same time points as in the TEM experiment above, PC12 cells were incubated with nanospheres and analysed immunohistochemically to determine whether nanospheres were located within lysosomes. All of the specimens showed negative colocalisation results of nanoparticles with intracellular compartments positive for LAMP-1 (lysosomal-associated membrane protein 1, which is present in the membranes of both late endosomes and lysosomes). This suggested that the membrane-bound aggregates of nanospheres seen in the TEM images were not yet in the process of being actively broken down by the cell, and were possibly contained in early endosomes. There is the possibility that the endosomes are not sufficiently acidic to cause rupture of vesicles and release of nanospheres in the cytoplasm. Also, nanospheres may not be functionalised with a sufficient density of PEI (or long enough PEI chains) to achieve membrane disruption.

Suppression of Caveolin- and Lipid Raft-Dependent Endocytosis does not Affect Nanosphere Internalisation

To determine whether caveolin- or lipid raft-dependent endocytosis was the mechanism of particle uptake, the level of fluorescence due to nanospheres in PC12 cells was quantified in which these uptake mechanisms were suppressed.

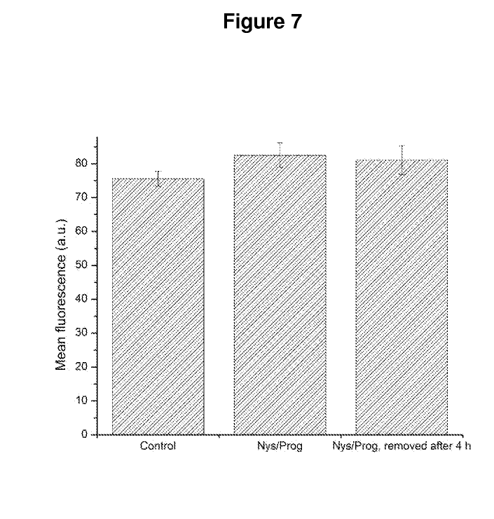

The addition of nystatin and progesterone to the cell culture resulted in sequestration of cholesterol and the inhibition of its synthesis, thereby disabling uptake by lipid rafts and caveolae. A change in particle uptake was not observed (as measured by intracellular fluorescence) when cells were exposed to nystatin (25 μg ml⁻¹) and progesterone (10 μg ml⁻¹) (FIG. 7). From this result, it was not possible to conclude that lipid rafts or caveolae were not involved in nanosphere endocytosis, but there was at least one other mechanism by which particles were internalised. The absence of visable clathrin pits and caveolin invaginations, and the presence of psueodopod-like structures suggested that macropinocytosis was a possible mechanism. Thus, it was concluded that uptake occurs mainly through macropinocytosis and clathrin- and caveolin-independent uptake. This was in contrast to smaller positively-charged particles (≦100 nm in size), which were apparently taken up by the clathrin route.

Magnetic Resonance can be Used to Follow Compartmentalisation within PC12 Cell

The multimodal imaging properties of the polymer nanospheres enabled further tests to be carried out characterising the nature of the particles in an in vivo system. The relaxation properties of the particles were measured to determine their usefulness as MRI contrast agents. Concurrently, results from the TEM study showed that particles became clustered over time within cells, presumably by active sorting. Clustering affects the relaxation rates of magnetic species, and thus, the relaxation properties of the particles were measured when they were in close proximity to one another in cells compared to when they were in the well-dispersed state.

The polymer nanospheres were first suspended in water and the r₁ and r₂ relaxivities measured. This was performed using Bruker minispec mq series instruments. The applicant found r₁=7.02 s⁻¹ mM⁻¹ and r₂=338 s⁻¹ mM⁻¹ based on iron content for the nanospheres, measured at 60 MHz (1.4 T).

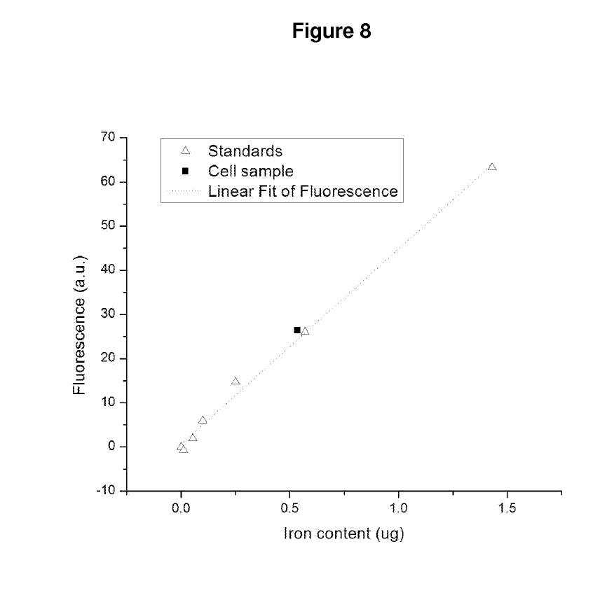

It is possible to follow the uptake of iron in cells if the relaxation rate is well defined, i.e., particles remain separate and in the same environment. As the nanospheres also contained a fluorescent probe, the iron content of samples could be determined as it was related to the observed fluorescence. A standard curve of fluorescence versus iron content was constructed with strong correlation (r=0.99), with which it was possible to determine the iron content of a cellular sample to within 10% error (FIG. 8).

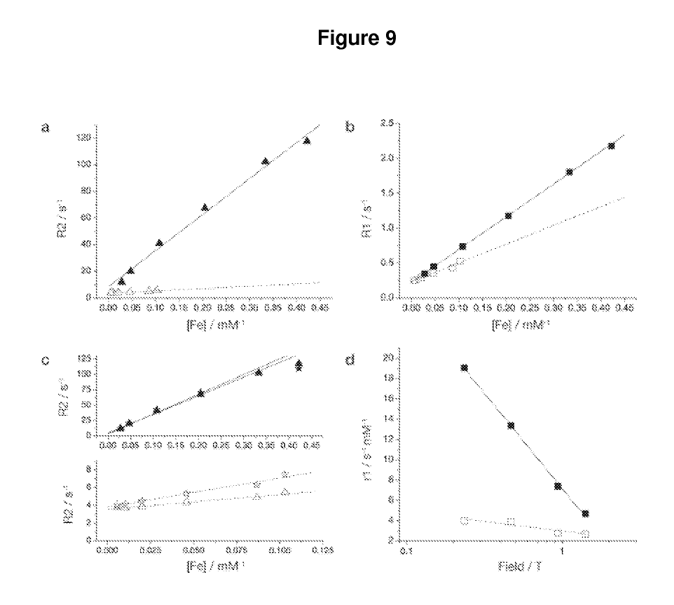

The change in behaviour upon clustering of the nanospheres, and whether the relaxation rate goes up or down, depends on particle size and magnetisation. Here it was determined that R₂ should decrease if particles were compartmentalised. The relaxivity of samples of cells incubated with nanoparticles for 72 hr suspended in agarose gel was measured. A decrease in both R₁ and R₂ was observed for particles in cells, which was the first indication that clustering in cells had occurred. There were several other indications of clustering; first, that R₂ depended on the duration of the echo measurement (T_{E}) for the nanospheres in cells, but not for well-dispersed nanospheres, and second, the different dependency of R₁ on field for the free nanospheres compared to nanospheres in cells (FIG. 9).

An approach was developed to directly image endocytosis and intracellular trafficking using fluorescent and magnetic polymer nanospheres. Polymer nanospheres were prepared from poly(glycidyl methacrylate) (PGMA) (modified with rhodamine B (RhB) dye) which was used to encapsulate magnetite (Fe₃O₄) nanoparticles. The epoxide groups on PGMA enabled anchoring of PEI chains by means of a simple ring-opening reaction (FIG. 1b). These nanospheres enabled a multimodal approach to directly assess how PEI mediates the cellular trafficking of nanoparticles, using correlated relaxometry, fluorescence spectroscopy and microscopy, and transmission electron microscopy. The polymer nanoparticles were synthesized using a nonspontaneous emulsification method, in which a binary solvent mixture containing both immiscible and soluble components was employed as the dispersed phase. This organic solution of the dye-modified polymer, also containing iron oxide nanoparticles (FIG. 1c), was emulsified with water in the presence of a surfactant. The polymer nanoparticles were characterized using transmission electron microscopy (FIG. 1d), dynamic light scattering (FIG. 1e), fluorescence spectrophotometry, and magnetometry. Analysis of nanospheres before and after PEI attachment revealed a small increase in average size and a large positive shift in the zeta potential distribution (FIG. 1e). The average particle diameter following PEI attachment was 160 nm (distribution 90-260 nm), and particles comprised approximately 3% PEI by weight. Nanospheres were magnetically separated from excess, unbound PEI.

### Toxicity of Nanospheres

The toxicity and transfection efficacy of PEI depend on both its molecular weight and structure (i.e., linear or branched). In this respect, the presence of free PEI may play a role in inducing cell dysfunction because both branched and linear configurations can bind to plasma membrane proteoglycans, compromise membrane integrity, and induce early necrotic-like changes within 30 min. In another respect, PEI-induced cytotoxicity has been related to the activation of a mitochondrially mediated apoptotic program involving channel formation in the outer mitochondrial membrane within 24 hr. In the present study, covalent grafting of PEI chains to the magnetic PGMA core allowed the removal of free PEI using magnetic separation, facilitating a clear determination of whether PEI-modified nanospheres were toxic or not.

The toxicity of the modified nanospheres was examined in rat pheochromocytoma neural progenitor (PC12) and retinal Müller glial cell lines (rMC-1), as well as primary rat hippocampal and cortical neuron cultures after 24 and/or 72 hr incubation. For the immortalized cultures, there was no decrease in viable cell numbers (p>0.05) for any of the tested concentrations (up to 250 μg mL¹). Similar results were observed in the primary neuron cultures, where a toxic effect (p≦0.05) was observed only at very high nanosphere concentrations (1000 μg mL⁻¹) that would be inappropriate therapeutically. The assay was verified by assessing trypan blue dye exclusion in PC12 cells. In line with previously proposed mechanisms of PEI toxicity, the applicant suggests two reasons for the observed lack of toxicity in the tested cell lines and primary cultures. First, the PEI chain length was shorter (1.2 kDa) than used in previous studies (25 and 750 kDa), and second, PEI was bound to the nanospheres and, as a result, may have been unable to interact with mitochondrial membranes. This investigation covered the time course of known cytotoxic changes.

### Nanosphere Uptake Monitored by Fluorescence

Nanosphere uptake was examined in more depth to further analyse intracellular trafficking and investigate why PEI-modified nanospheres were not toxic. When nanospheres were incubated with PC12 cells, nanospheres with PEI modification were taken up rapidly, within minutes, while those without PEI modification were not associated with cells even after 3 days. Nanospheres that were internalized presented a punctate distribution and were excluded from the nucleus (FIG. 3a-d). To examine the dynamics of endocytosis, the applicant used live cell confocal imaging to monitor fluorescence of nanospheres in PC12 cells for periods of 24 and 72 hr (FIG. 3e). In time-lapse experiments, using a single confocal slice through the cells, the applicant observed a linear increase in fluorescence per cell with time (FIG. 3f). Similar results were obtained with primary cortical and hippocampal neuronal cultures, with a greater intensity of fluorescence per cell in cortical cells. There was no apparent correlation between the intensity of fluorescence indicating nanosphere uptake and the viability of the various cell cultures following treatment. The result in PC12 cells was also confirmed using a spectrofluorometric plate reader to measure intensity over large samples of cells.

### Endocytosis of Nanospheres Determined by Drug Inhibition and Electron Microscopy

There are many distinct endocytic pathways that coexist in mammalian cells that regulate the entry of a wide range of different sized moieties from ions to macromolecules, pathogens, and drugs. It follows that polymeric nanoparticle drug delivery systems are also subject to the same selectivity. Uptake of nanoparticles by endocytosis can occur via a number of different routes depending on the cell type and the nature of the cargo (nanoparticle size and surface charge). Pinocytosis, or fluid-phase uptake, has often been reported as a common route for uptake of positively charged macromolecules, comprising macropinocytosis (for particles >1 μm), clathrin-mediated endocytosis (≦120 nm), and caveolin-mediated endocytosis (≦90 nm). In the case of PEI/DNA polyplexes, endocytosis is initiated when polyplexes bind to syndecans, which are negatively charged heparan sulfate proteoglycans (HSPGs) in cell membranes. Syndecan clustering around the particle triggers cytoplasmic binding of these transmembrane proteins to actin filaments through linker proteins, which subsequently supports polyplex uptake through endocytic vesicles. More recently, a distinct fluid-phase pathway, independent of clathrin and caveolin, has been identified to contribute to the uptake of PEI-25/DNA polyplexes of size ≧150 nm, and importantly, macropinosomes have been reported to have a higher propensity to deliver PEI-25/DNA cargo than endosomes. Consequently, multiple pathways of intracellular trafficking, including macropinocytosis, should be considered in the analysis of PEI-mediated endocytosis of our nanospheres. The internalization of nanospheres was investigated in PC12 cells following treatment with well-known inhibitors of clathrin-mediated endocytosis (chlorpromazine), lipid raft- and caveolin-mediated endocytosis (nystatin/progesterone), and macropinocytosis (N,N-dimethylamiloride). The applicant observed no statistically significant reductions in uptake (p>0.05) for any of these drugs either individually or in combination. As the uptake of PEI-modified nanospheres was not significantly inhibited by treatment with selected drugs, macropinocytic and clathrin- and caveolin-mediated endocytic routes are probably not required for uptake of polymer nanospheres. Furthermore, the result implies that there is at least one other possible mode of entry for these nanoparticles. It is important to note that the use of inhibitors to identify uptake pathways is not conclusive, because such treatment may up-regulate a pathway that is usually silent. Up-regulation of nascent pathways has cell-wide consequences in terms of signalling, lipid and protein distributions, membrane tension, and stress responses. Therefore, electron microscopy was used as an additional means of assessing endocytosis as well as the intracellular itinerary of the internalized PEI-modified nanospheres.

Transmission electron microscopy was used to image cellular processes involved in endocytosis and cellular trafficking after 3, 6, 12, 24, and 72 hr exposure to nanospheres (FIG. 5). In these images, the polymer nanospheres appear as light circles, while the encapsulated iron oxide nanoparticles are much smaller and electron dense, as in FIG. 1e. PEI-mediated endocytosis involved adsorption of nanospheres to the cell surface, perhaps suggesting that endocytosis is triggered by interaction with syndecans on the cell membrane as for cationic polyplexes. The number of nanospheres associated with the cell surface increased with time. Nanosphere uptake was accompanied by protrusions and invaginations of the plasma membrane (FIG. 5a), characteristic of macropinocytosis, and also tubular invaginations characteristic of clathrin and caveolin-independent endocytosis extending 0.5-2 μm into PC12 cells (FIG. 5b). These observations accord with the results of the chemical inhibition experiment, as nanosphere uptake was not suppressed when macropinocytosis and clathrin- and caveolin-dependent endocytosis were inhibited.

Individual nanospheres were observed throughout the cytoplasm of PC12 cells within 3 hr (FIG. 5c). After 6 hr, nanospheres appeared to have a greater proximity to one another, forming loose clusters that were occasionally membrane-bound. After 12 hr, loose clusters were frequently observed near vesicles enclosed by multiple membranes, and by 24 hr, these clusters appeared to be mostly membrane-bound. The membrane-bound nanosphere clusters themselves were often grouped together, and in some instances membranes were apparently in the process of fusion (FIG. 5d). By 72 hr, the clusters were larger (200-500 nm) and comprised several smaller membrane-bound packages that were grouped together and enclosed by one or more membranes (FIGS. 5d,e). These observations suggested that nanospheres were being captured within endosomes, and the presence of multivesicular bodies indicated endosome maturation.

Immunohistochemistry Shows that Nanospheres are not in Lysosomes

Lysosomes constitute the final degradative stage in the endocytic journey, vary in size and appearance, and are usually associated with membrane whorls similar to those we observed ultrastructurally. Using the same time points as the TEM experiment, PC12 cells were incubated with nanospheres and analysed immunohistochemically for lysosomal-associated membrane protein 1 (LAMP-1), a marker of both late endosomes and lysosomes. We did not observe colocalization of nanospheres with LAMP-1 immunopositive intracellular compartments.

It appears that the lack of observed toxicity of the nanospheres is consistent with observations of nanosphere compartmentalization (and increase in the size of these aggregates), rather than osmotic swelling. In the case of polyplexes, buffering of endosomal compartments by polyamines results in increased entry of H [symbol] , with concomitant Cl⁻ uptake, which in turn promotes osmotic swelling and endosomal leakage or lysis. This destructive process is the mechanism whereby the DNA polyplex escapes lysosomal capture and so accesses the nucleus with gene delivery and resultant expression. The process is rapid, with release via osmotic swelling occurring within 4 hr and DNA expression peaking at 24 hr. Osmotic swelling or signs of endosomal escape was not observed which correlates with a lack of toxicity within 24 hr. This suggests a sequence in which smaller nanosphere clusters fused to form larger aggregates, but were not contained in lysosomes within 72 hr.

### Relaxometry can be Used to Follow Nanosphere Endocytosis

The observation of compartmentalization of nanospheres in individual PC12 cells by TEM was confirmed in a larger population of cells using relaxometry. This analysis was made possible by the superparamagnetic iron oxide nanoparticles contained within the nanospheres. Superparamagnetic nanoparticles are effective contrast agents for magnetic resonance (MR) imaging, as they possess a large magnetic moment and are free to align with an applied magnetic field. The resultant microscopic field gradients diphase nearby protons, shortening both the longitudinal relaxation time T1 and the transverse relaxation time T2. T1 is also reduced via other relaxation mechanisms. The corresponding relaxation rates, R1 and R2, are influenced by the local concentration of nanoparticles, the applied field strength, and the environment in which the nanoparticles interact with surrounding protons. In particular, the relaxation induced by iron oxide nanoparticles is known to depend on their compartmentalization in macrophages, lymphocytes, oligodendrocytes, human neural stem cells, and mesenchymal stem cells.

The applicant analysed relaxation rates in PC12 cells after 72 hr exposure, by which time PEI-modified nanospheres were bound within intracellular compartments. We compared relaxation rates of nanospheres that had been endocytosed by PC12 cells with those of free nanospheres, both of which were held in agarose gels. The transverse relaxivity of free nanospheres at 1.4 T was r2_{free}=322±26 mM⁻¹ s⁻¹ with a 19-fold reduction in PC12 cells (r2_{cell}=16.7±1.2 mM⁻¹ s⁻¹; FIG. 9a). There was a 2-fold decrease in the longitudinal relaxivity r1 (r1_{free}=4.70±0.05 mM⁻¹ s⁻¹, r1_{cell}=2.70±0.26 mM⁻¹ s⁻¹; FIG. 9b). It is predicted by the applicant that the decrease in r2 on compartmentalization of nanospheres is attributed to the behaviour of the aggregates as a micrometric nanosphere. The larger effective diameter shifts the compartmentalized nanoparticles into the echo-limited proton relaxation regime. In this echo-limited environment, the perturbations in the magnetic field experienced by the water protons become effectively static. Hence, the refocusing pulses become efficient, reducing r2 compared to the dispersed nanoparticles and introducing a significant dependency on the echo spacing (FIG. 9c). Furthermore, the dramatic dependence of r1 on magnetic field strength (FIG. 9d) is also consistent with clustering of the nanoparticles and similar to theoretical predictions and observations. We attribute the reduction in r1 to the less rapid innersphere exchange of water molecules inside the cell as well as a reduced molecular exchange with bulk water through a “membrane-shielding” effect.

Using fluorescence intensity as a measure of iron content, the applicant expects that the state of clustering within cells could be measured over time using relaxometry. This advantage of the multimodal nanospheres will be helpful in following endolysosomal sorting not only in vitro but also on a larger scale in vivo using live fluorescence imaging in conjunction with MRI. A standard curve of fluorescence versus iron content was constructed with strong correlation (r=0.9970), from which we were able to determine the iron content of a cellular sample to within 10% error.

### Conclusion

Understanding the routes and being able to follow aspects of the endocytosis of polymer nanoparticles is an important area that has received little attention. In this study, insights were gained into the way polymer nanospheres with a size around 170 nm are taken up by PC12 cells. These particles can be loaded with drugs for sustained release. It was shown that these particles have very little toxicity in neuronal cells and related cell lines, and that the incorporation of two imaging modalities allows for further characterisation of particle behaviour than either technique alone.

The nanospheres used in the present example allow direct visualization by electron and fluorescence microscopy, as well as the ability to examine compartmentalization after endocytosis by relaxometry. The magnetic properties also enabled removal of excess PEI, enabling examination of PEI-mediated endocytosis without the confounder of toxicity of free PEI. Endocytosis involved a clear sequence of events: interaction of nanoparticles with the cell membrane induced membrane ruffling and tubular invagination, characteristic respectively of unregulated/unselective macropinocytosis and clathrin- and caveolin-independent endocytosis, followed by time-dependent intracellular clustering within lamellar envelopes. The nanosphere architecture thus offers a broad scope for delivery of a wide range of agents to intracellular compartments. The findings we have presented will assist in the design and synthesis of next-generation nanoparticles for site-specific drug delivery.

### Materials and Methods

### Materials

All chemicals were purchased from Sigma-Aldrich unless otherwise stated: benzyl ether (99%), chlorpromazine hydrochloride (98%), 5-(N,N-dimethyl)amiloride hydrochloride, iron(III) acetylacetonate (97%), nystatin, oleic acid (BDH, 92%), oleyl amine (70%), Pluronic F-108, polyethylenimine (50% solution, Mn 1200, Mw 1300), progesterone (99%), rhodamine B (Kodak®, 95%), and 1,2-tetradecanediol (90%) were used as received. All tissue culture reagents were purchased from Invitrogen® unless otherwise stated: B27, bovine serum albumin (Aldrich®), DMEM, fetal bovine serum, L-glutamine 200 mM, GlutaMAX 100x, horse serum, MEM, Neurobasal, nonessential amino acids (NEAA) 10x penicillin/streptomycin, poly(_{L}-lysine) (Aldrich®), RPMI1640, sodium pyruvate 100x, and trypsin/EDTA. Samples were mounted for light microscopy using Fluoromount-G (Southern Biotech®).

### Preparation of Iron Oxide

Fe₃O₄ was synthesized by the organic decomposition of Fe(acac)₃ in benzyl ether at 300° C., in the presence of oleic acid, oleyl amine, and 1,2-tetradecanediol.

### Synthesis of RhB-Modified PGMA

Glycidyl methacrylate was polymerized in methyl ethyl ketone (MEK) to give PGMA (Mw=250 000 g mol⁻¹), using azobisisobutyronitrile as initiator. The polymer was purified by multiple precipitations from MEK solution using diethyl ether. To attach the dye to the polymer, a solution of rhodamine B (RhB, 20 mg) and PGMA (100 mg) in MEK (20 mL) was heated to reflux under N₂ for 18 hr. The solution was reduced in vacuo before the modified polymer was precipitated with diethyl ether (20 mL). The polymer was redissolved in MEK and precipitated with ether twice to remove ungrafted RhB.

### Polymer Nanosphere Preparation

Nanoparticles were prepared using a nonspontaneous emulsification route.

The organic phase was prepared by dispersing iron oxide nanoparticles (20 mg) and dissolving PGMA-RhB (75 mg) in a 1:3 mixture of CHCl₃ and MEK (6 mL). The organic phase was added dropwise, with rapid stirring, to an aqueous solution of Pluronic F-108 (1.25% w/v, 30 mL), and the emulsion was homogenized with a probe-type ultrasonicator at low power for 1 min. The organic solvents were allowed to evaporate overnight under a slow flow of N₂. Centrifugation at 3000 g for 45 min removed large aggregates of iron oxide and excess polymer. The supernatant was removed to a 50 mL flask containing PEI (50 wt % solution, 100 mg) and heated to 80° C. for 18 hr. The magnetic polymeric nanospheres were collected on a magnetic separation column (LS, Miltenyi Biotec®), washed with water (2×1.5 mL), and then flushed with water until the filtrate ran clear. The resulting concentrated particle suspension was aliquoted (ca. 10×500 μL) and stored at 4° C. for quantification by lyophilisation, analysis, and subsequent use. Nanospheres were sterilized by UV irradiation.

### Cell Culture

Rat pheochromocytoma cells (PC12) were obtained from the Mississippi Medical Center (Jackson, Miss.), cultured in poly-(_{L}-lysine)-coated polystyrene flasks in a humidified atmosphere containing 5% CO₂ at 37° C., and maintained in RPMI1640 medium containing horse serum (10% v/v), fetal bovine serum (5% v/v), penicillin/streptomycin (100 U mL⁻¹, 100 μg mL⁻¹), L-glutamine (2 mM), nonessential amino acids (100 μM), and sodium pyruvate (1 mM). For experiments, cells were seeded in 24- or 96-well plates or on glass coverslips coated with poly-(_{L}-lysine) at a cell density of (0.5-2)×10⁵ mL⁻¹ and incubated for 24 hr prior to experiments. Cells were not differentiated. Retinal Müller cells (rMC-1) were cultured in uncoated polystyrene dishes in DMEM high-glucose medium containing fetal bovine serum (10%), penicillin/streptomycin (50 U mL⁻¹, 50 μg mL⁻¹), and L-glutamine (2 mM). Cells were seeded at 1×10⁵ mL⁻¹ and incubated for 24 hr prior to experiments. Hippocampal and cortical neuronal cultures were prepared as follows and maintained for 7-14 days prior to experiments.

Rat pups (Sprague-Dawley, P1; or PVG, P1-P3) were placed in a CO₂ atmosphere and rapidly decapitated or anesthetized with xylazine/ketamine (Ilium xylazil and Ketamil, Troy Laboratories, 10 and 50 mg kg⁻¹, respectively, ip) and euthanized with Euthal (pentobarbitone sodium 850 mg kg⁻¹, phenyloin sodium 125 mg kg⁻¹; ip). Brains were removed to a dish containing dissociation media [DM: in H₂O; MgCl₂, 5.8 mM; CaCl₂, 2.5 mM; HEPES, 1.6 mM; phenol red, 8 mg L⁻¹; Na₂SO₄, 90 mM; K₂SO₄, 18.75 mM] on ice, from which hippocampal and cortical tissue was isolated and removed to DM on ice. DM was removed, and prewarmed enzyme solution [ES: DM, 10 mL; papain, 200 U; L-cysteine, 1.6 mg] was added to the tissue, which was incubated at 37° C. for 25 min with gentle shaking every 5 min. The supernatant was replaced with heavy inhibitor [HI: DM, 12 mL; trypsin inhibitor, 120 mg; bovine serum albumin, 120 mg] for 2 min, then light inhibitor [LI:DM, 9 mL; HI, 1 mL] for 2 min, and then with plating media [PM: in Minimum Essential Medium; fetal bovine serum, 10% v/v; glucose, 20 mM; pen/strep, 20 U mL⁻¹, 20 μg mL⁻¹; GlutaMAX, 2 mM; sodium pyruvate, 1 mM] to a suitable dilution for cell counting. The tissue was triturated until a homogeneous cell suspension was obtained, and cells were further diluted and plated on glass coverslips coated with poly-(_{L}-lysine) (10 μgmL⁻¹) overnight at 37° C. Four hours later, PM was replaced with feeding media [FM: in Neurobasal; glucose, 12 mM; pen/strep, 20 UmL⁻¹, 20 μgmL⁻¹; GlutaMAX, 0.5 mM; B27, 2% v/v; 2-mercaptoethanol, 25 μM]. Half of the culture media was replaced twice weekly. Morphological assessment of primary cultures indicated that approximately 95% of cells were neuronal due to the initial serum-free conditions (n=1000 cells assessed).

### Cell Viability Measurements

Viability was measured using a Live/Dead cell kit (Invitrogen®). Cells were incubated for 24 hr before the cell media was replaced with nanoparticle suspensions of different concentrations in media. After a further 24 or 72 hr, the nanoparticle suspension was removed, the cells were washed once with PBS, and 100 μL of Live/Dead reagents was added (calcein AM, 1 μM; ethidium homodimer-1, 3 μM). After 30 min, images were recorded using an inverted fluorescence microscope at 20× magnification (Olympus IX-71, Olympus IX-81). Four images were recorded from each well at consistent locations for all wells and all experiments, and live and dead cells were counted. The trypan blue dye exclusion assay was used as an additional measure of cell viability, following similar treatment procedures. Cells were detached using trypsin-EDTA, resuspended in 100-200 μL of cell media, combined with trypan blue, and counted using a hemocytometer or an automated cell counter (Invitrogen® Countess).

### Endocytic Inhibition Study

PC12 cells were plated in 96-well plates as above. Solutions of chlorpromazine (10 μg mL⁻¹), nystatin, and progesterone (25 and 10 μgmL⁻¹, respectively), or 5-(N,N-dimethyl)amiloride (50 μM) were prepared in complete media; progesterone was diluted from a 2 mg mL⁻¹ stock in EtOH. Cells were incubated for 1 hr with inhibitors, and then the drugs were placed together with nanospheres (10 μg mL⁻¹). After 3 hr, wells were washed with PBS and fluorescence was quantified (BMG® FluoStar Optima).

### Immunohistochemical Analysis

PC12 cells were grown as above, incubated with nanospheres (1-10 μg mL⁻¹) for 3, 6, 12, 24, or 72 hr, and fixed in paraformaldehyde (4%). Fixed cells were incubated in PBS containing Triton X-100 (0.2%) and blocked (7.5%) for 10 min, then incubated in the same solution containing anti-LAMP1 (Abcam®, 1:1000), anti-β-III-tubulin (Tuj-1, Chemicon, 1:500), and/or anti-G FAP (Dako®, 1:1000) overnight. Antibodies and nuclei were visualized by incubation with Alexa 488 and/or Alexa 647 (Molecular Probes®, 1:400) and Hoechst 33342 (Sigma®, 1 μg mL⁻¹) for 1-2 hr. Images were captured by confocal microscopy (Leica® TCS SP2, Nikon® A1Si).

### Preparation of TEM Samples

PC12 cells were grown on 8 mm² or 1.2 mm diameter 50.8 μm Aclar film for chemical fixation or cryopreservation, respectively. The films were attached to the surface of 12-well culture plates by spot-welding and then UV sterilized prior to the addition of cells. PC12 cells were plated as above and treated with nanospheres (10 μg mL⁻¹) for 3, 6, 12, 24, or 72 hr prior to fixation or cryopreservation. Following chemical fixation (2.5% glutaraldehyde in PBS, pH 7.4), samples were rinsed with PBS and postfixed (1% OsO₄) prior to dehydration in a graded series of ethanol. Alternatively, cryopreservation was achieved by high-pressure freezing (Leica® EM PACT2) after dipping sample discs in cryoprotectant (2 mg mL⁻¹ low-gel agarose in cell media at 37° C.). Frozen samples were placed in freeze-substitution media (1% osmium tetroxide, 0.2% uranyl acetate, and 3% water in acetone) and gradually brought to room temperature in a freeze-substitution unit (Leica® EM AFS2). All specimens were embedded in Procure-Araldite before sections (80-120 nm) were cut and collected on uncoated 200-mesh copper grids. Grids of conventionally processed specimens were stained with uranyl acetate and lead citrate prior to observation, while grids of high-pressure frozen specimens were unstained. Iron oxide and nanosphere samples were prepared for TEM by deposition on carbon-coated copper grids. All TEM images were obtained at 120 kV (JEOL JEM-2100).

### Relaxometry

Relaxivity data were measured using four Minispec mq series instruments (Bruker®) operating at 0.23, 0.46, 0.92, and 1.41T. A Carl-Purcell-Meiboom-Gill (CPMG) spin echo sequence was used to measure T2. The echo spacing was 2 ms for the short TE measurements (1000 echoes) and 10 ms for the long TE measurements (200 echoes), with a repetition time of 10 s for both. An inversion recovery (IR) sequence was used to measure T1 using 10 inversion times (TI) logarithmically spaced between 50 and 5000 ms. One 75 cm² flask of PC12 cells was incubated with polymer nanospheres for 72 hr at a concentration of 50 μg mL⁻¹. Cells were detached from the substrate, fixed in 4% paraformaldehyde, and counted before dilutions were prepared for relaxometry measurements. The samples were suspended in 0.5% agarose gel, and data were recorded at 37.5° C. The iron content of the samples was determined by ICPAES after acid digestion (5 mL).

### Example 2

### Intracellular Release of a Calcium Channel Blocker from Multimodal Nanoparticles

### Results and Discussion

Protecting vulnerable neurons and glia from damage that occurs secondary to neurotrauma is one of several requirements for successful therapy. Deregulation of calcium ion homeostasis is associated with secondary degeneration and release of excitatory amino acids, initiating cascades that lead to increased mitochondrial production of reactive oxygen species and cell death. Calcium channel blockers have been shown to prevent calcium influx in vitro and protect against secondary injury effects in vivo. In this work, lomerizine, a calcium channel blocker with limited aqueous solubility, was encapsulated in multifunctional polymer nanospheres. The protective effect of lomerizine, that is, preventing calcium influx, was demonstrated by measuring the intracellular calcium concentration in PC12 cells exposed to glutamate. The results show that targeted intracellular delivery of lomerizine using labeled nanoparticles may be a feasible approach to deliver sustained doses of lomerizine to CNS injury sites.

Neurotrauma describes a physical injury to the CNS, usually acute and caused by mechanical insult. The initial impact and forces cause deformation of tissue, producing direct neuronal and vascular injury that may be focal or diffuse. In addition to the primary injury, post-traumatic edema, ischemia, inflammation, neurotransmitter accumulation (particularly of the excitatory amino acid glutamate) and changes in intracellular ion concentrations (including [Ca²⁺]) may be toxic to neuronal and glial cells. These indirect effects and resultant cell death (together termed secondary degeneration) can lead to secondary brain damage and post-traumatic epilepsy, as well as further loss of sensory, motor and autonomic function. The complications of neurotrauma are considered more amenable to treatment than the injury itself, because the severity of the primary injury cannot be modulated by drugs after the event. Thus, a key objective is to prevent or minimize the various causes of secondary damage. Secondary degeneration is initiated within minutes of injury, and develops over several weeks following the initial insult. There is an important window of therapeutic opportunity during this time to protect vulnerable cells that were spared by the initial event. Even rescuing a small proportion of neurons can have significant benefits in maintaining function.

Secondary degeneration is associated with alterations in flux of intracellular calcium in neurons and glia. Loss of Ca²⁺ homeostasis leads to increased production of reactive oxygen species by mitochondria, initiating biochemical cascades that can activate calpains and caspases. The activation of these enzymes leads to breakdown of proteins, lipids, and nucleic acids, contributing to apoptosis and delayed cell death. Increase of [Ca²⁺]ᵢ can occur by several mechanisms, including release from buffering proteins, release from intracellular stores, or as a result of influx through Ca²⁺ channels residing in the plasma membrane. Of the routes for Ca²⁺ influx, N-methyl-d-aspartate (NMDA) receptors constitute one of the most significant entry mechanisms, because glutamate-activated NMDA receptors are highly permeable to Ca²⁺. NMDA receptor antagonists have therefore been identified as a therapeutic target for neuroprotection. While these drugs showed initial promise in animal studies of neurotrauma, the results did not translate to clinical trials, and blocking glutamate receptors impinges on normal brain function and causes side effects. In addition to entry through NMDA receptors, calcium can also enter neurons or glia through acid-sensing ion channels (ASICs), α-amino-3-hydroxy-5-methyl-4-isoxazolepropionate (AMPA) receptors, P2X₇ purinergic receptors, or voltage-gated calcium channels (VGCCs). Drugs that prevent calcium influx through these routes are neuroprotective in a range of injury models.

Lomerizine (KB-2796, 1-[bis(4-fluorophenyl)methyl]-4-(2,3,4-trimethoxybenzyl)piperazine dihydrochloride) is a calcium channel blocker that selectively blocks L- and T-type Ca²⁺ channels, but not N-type channels. Lomerizine has no reported affinity for NMDA, kainate or AMPA binding sites, so neuroprotective benefits of the drug are probably specifically due to VGCC blockade rather than glutamate receptor antagonism. Additionally, lomerizine may reduce formation of reactive oxygen species in dissociated cerebellar neurons. In accordance with its calcium channel-blocking abilities, lomerizine protects against H₂O₂-induced death, glutamate-induced toxicity, and glutamate-induced cell death in rat hippocampal neuron cultures; protects against hypoxia in rat retinal ganglion cell cultures; improves recovery after ischemic spinal cord damage in rabbits; protects neurons in visual centers of the brain after retinal damage by intravitreal injection of NMDA in mice; protects against cerebral hypoxia/ischemia and retinal ischemia/reperfusion in rats; and provides limited protection against secondary degeneration following partial optic nerve injuries in rats.

Administration of lomerizine for neuroprotection has some drawbacks. Regular doses of lomerizine are required, because the half-life of the drug is short (elimination half-life 108 hr in humans) compared to the optimal duration of treatment. Also, 24 hr after oral administration of lomerizine in rats, 51.7% of the dose was excreted in the feces, and 4.1% excreted in urine, suggesting that lomerizine uptake by the gastrointestinal system is not particularly efficient. In previous in vivo studies of neuroprotection, therefore, lomerizine was administered orally at least once a day at doses of 10-30 mg kg⁻¹ in rats and rabbits. A delivery system using nanoparticles could assist in improving the bioavailability of the drug, and also enable sustained release over time.

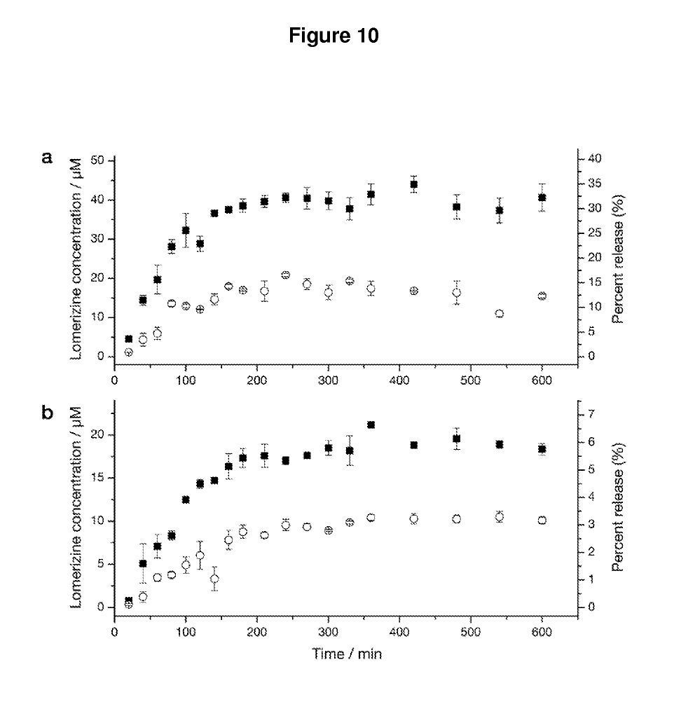

In this report, we use magnetic and fluorescent polymer nanospheres for intracellular delivery of lomerizine. The uptake of these nanospheres, shown in FIG. 1c, has been demonstrated in PC12 rat neural progenitor cells. PEI functionalization is a requisite for cellular uptake of these nanospheres in PC12 cells (FIGS. 3a, 5d). Nanosphere preparations are detailed in supporting information. The mean hydrodynamic diameter of the unmodified nanospheres was 210 nm (FWHM 120 nm) as measured by dynamic light scattering (FIG. 1e), with a small increase in size associated with PEI attachment. The encapsulation efficiency of lomerizine in nanospheres was (7.1±1.3) %. Drug release from nanospheres into phosphate-buffered saline (PBS) was assessed at physiological and slightly acidic pH (FIG. 10). The rate and quantity of lomerizine released from polymer nanospheres decreased with increasing pH; at normal physiological pH (7.4) the release of lomerizine was below the detectable limit at all time points. In these experiments, the concentration of lomerizine in the sink reached a maximum level after several hours; this may reflect saturation of the sink solution that would be unlikely to occur in vivo. The extent of the lomerizine release from the modified nanospheres was reduced by PEI on the particle surface. This was perhaps a result of steric hindrance or electrostatic interactions between the drug and PEI, both of which carry a positive charge in acidic solution.

We have previously demonstrated that PEI-modified polymer nanospheres localize in endosomes within 24 hr (FIG. 1d). The pH in early endosomes is mildly acidic and falls below 5 as endosomes mature, so release of lomerizine from nanospheres could be triggered under these conditions. Here, we administered lomerizine to PC12 cells using nanospheres, and measured intracellular calcium concentrations after glutamate challenge to see if intracellular release of lomerizine could be employed for neuroprotection. PC12 cells express both N- and L-type VGCCs, but not T-type channels, and in undifferentiated PC12 cells, glutamate exposure (1-10 mm) over 24 hr causes oxidative stress and increased [Ca²⁺]ᵢ. Glutamate-associated toxicity in PC12s has been shown to be independent of typical NMDA receptors, and the PC12 cell line is therefore suitable for assessing L-type calcium channel blockade by lomerizine.

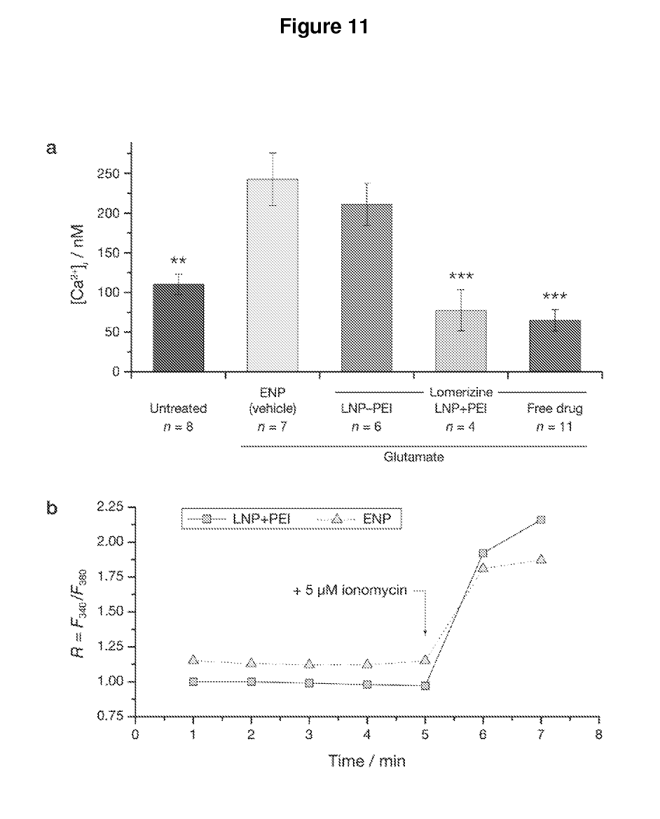

Cells receiving nanosphere treatments were incubated with nanospheres containing lomerizine, with or without modification with PEI (LNP+PEI and LNP−PEI respectively), or nanospheres containing no lomerizine but with PEI modification (ENP). These treatment groups were compared with cells treated with lomerizine only (i.e., the free drug) at 1 In all cases, nanospheres were added at 25 μg mL⁻¹, which was estimated to yield a concentration of lomerizine similar or lower than the free drug, based on the data in FIG. 10. The results are shown in FIG. 11.

Compared to resting cells (“untreated”), [Ca²⁺]ᵢ was significantly increased in cells that were treated with empty nanospheres (ENP) and exposed to glutamate (p≦0.01). Lomerizine delivered using PEI-modified nanospheres (LNP+PEI), or added as the free drug dissolved in 0.01% DMSO, resulted in a significant (p≦0.001) decrease in [Ca²⁺]ᵢ in the presence of glutamate; both routes were equally effective. Nanospheres containing lomerizine but without PEI modification (LNP−PEI) did not reduce [Ca²⁺]ᵢ (p>0.05) compared to cells treated with empty nanospheres. This was presumably because these LNP−PEI nanospheres were not internalized by cells and were not exposed to the acidic endosomal environment. Taken together, these results suggest that lomerizine release from nanospheres requires a drop in pH and that PEI-modified nanospheres may be a suitable vehicle for intracellular release of this drug.

Internalization of polymer nanospheres is not a requirement for lomerizine release, however, as release of the drug will occur in any environment with lowered pH. After CNS injury, vascular interruption causes a switch to anaerobic glycolysis in the brain; this results in a decrease in pH in the intercellular space known as acidosis. The pH falls about 1 unit during ischemia, and can drop below pH 6.0 during severe ischemia or under hyperglycemic conditions. In this situation, we would expect lomerizine release from nanospheres with the dose being dependent on the extent of injury. Therefore, we suggest that the nanospheres we have described will be useful in either their PEI-modified or unmodified forms, and that intracellular delivery or release into the intercellular space could be achieved.

### Conclusion

The use of multifunctional polymer nanoparticles has been demonstrated to facilitate intracellular delivery of a calcium channel blocker in vitro. Release of lomerizine from nanospheres will likely assist in improving the bioavailability of this drug and may provide a means for extended release under slightly acidic conditions. Lomerizine alone is not sufficient to prevent secondary damage arising as a result of neurotrauma and it is likely that combinatorial therapies will be required to target not only the neurochemically-induced changes, but also to initiate and provide a permissive environment for axonal regrowth. A nanoparticle-based approach could be beneficial for this purpose with potential delivery of multiple therapeutic strategies simultaneously.

### Materials and Methods

### Drug Loaded Polymer Nanosphere Preparation

Nanoparticles were prepared using a non-spontaneous emulsification route. The organic phase was prepared by dispersing iron oxide nanoparticles (15 mg) and dissolving PGMA (75 mg) and lomerizine dihydrochloride (10 mg) in a 1:3 mixture of CHCl₃ and MEK (6 mL). The organic phase was added dropwise, with rapid stirring, to an aqueous solution of Pluronic F-108 (1.25% w/v, 30 mL) in Tris buffer (10 mm) at pH 9.0. The resulting emulsion was homogenized with a probe-type ultrasonicator at low power for 1 min. The organic solvents were allowed to evaporate overnight under a slow flow of N₂. Centrifugation (3000 g, 45 min) removed large aggregates of iron oxide and excess polymer. For samples with PEI modification, the supernatant was removed to a 50 mL flask containing PEI (50 wt % solution, 100 mg) and heated to 80° C. for 18 hr. The magnetic polymeric nanospheres were collected on a magnetic separation column (LS, Miltenyi Biotec), washed with Tris buffer (2×1.5 mL), and then flushed with Tris buffer until the filtrate ran clear. The resulting concentrated particle suspension was aliquoted for quantification by lyophilization and subsequent use. Nanospheres were centrifuged, resuspended in PBS, and sterilized by UV irradiation immediately before use in tissue culture experiments.

### Drug Release

Experiments were performed at 37±0.1° C. in phosphate buffered saline (PBS) at pH 5.0, 6.0 and 7.4. Nanospheres (10 mg) were dispersed in PBS (10 mL) and the sinks were sampled in duplicate over 10 hr as follows. Aliquots of 150 μL were transferred to filter tubes (Millipore®, Amicon® Ultra-0.5, 50 kDa cut-off), centrifuged (17,000 g, 5 min), and analysed by RP-HPLC using a similar procedure to H. Waki, and S. Ando, J. Chromatogr. B 1989, 494, 408. The measurements were run on a Waters 2695 separations module coupled with Waters 2489 UV/Vis detector and C₁₈ column (150×4.60 mm, 5 μm, 25±5° C.), using isocratic elution with 69:31 acetonitrile/0.1% potassium phosphate buffer (pH 6) at 10.0 mL min⁻¹, monitoring the eluent at 210 nm. Each sample was run for 13 min and the integrated area of the largest peak between the retention time 9-10 min was used to calculate the lomerizine concentration. No fresh PBS was introduced into the sinks. Lomerizine concentrations were determined from a standard curve and reported as mean values±SE. The limit of detection for lomerizine in water at 210 nm was 0.1 μg mL⁻¹.

### Cell Culture

Rat pheochromocytoma cells (PC12) were obtained from the Mississippi Medical Center (Jackson, Miss.), cultured in poly(l-lysine)-coated polystyrene flasks in a humidified atmosphere containing 5% CO₂ at 37° C., and maintained in RPMI1640 medium containing horse serum (10%), fetal bovine serum (5%), penicillin/streptomycin (100 U mL⁻¹, 100 μg mL⁻¹), 1-glutamine (2 mm), non-essential amino acids (100 μm) and sodium pyruvate (1 mm). Cells were not differentiated with NGF.

### Intracellular Calcium Quantification

For experiments, cells were seeded in 24-well plates containing 10 mm glass coverslips coated with poly(l-lysine) at a cell density of 5.0×10⁴ mL⁻¹. Cells were allowed to attach for 12 hr and then growth media was replaced with treatments in media (lomerizine, 1 μm; empty PGMA nanoparticles+PEI (ENP), 25 μg mL⁻¹; nanoparticles containing lomerizine±PEI (LNP±PEI), 25 μg mL⁻¹). Lomerizine was dissolved in DMSO at a non-toxic final concentration of 0.01%. After 24 hr incubation, glutamate was added to a final concentration of 10 mm. After a further 24 hr, [Ca²⁺]ᵢ was quantified as follows. Coverslips were transferred into HBS solution containing (in mm) NaCl 140, KCl 5.4, CaCl₂ 2.5, MgCl₂ 0.5, HEPES 5.5 and glucose 11, pH adjusted to 7.4, supplemented with Fura-2 AM (1 μm) and incubated at 37° C. for 2 hr. Cells were then imaged (ex 340/380 nm, em 510 nm). The ratio of the emissions at the two wavelengths is denoted R=F₃₄₀/F₃₈₀. Fluorescent data were measured on a Hamamatsu Orca ER digital camera attached to an inverted Nikon TE2000-U microscope, and analysed using Metamorph v6.3. Calibrations were performed in 8 cells. Cells were exposed to 5 ionomycin to achieve a steady state maximum (Rₘₐₓ). Media was then replaced with Ca²⁺ free HBS containing 3 mm EGTA to achieve a steady state minimum (Rₘᵢₙ). [Ca²⁺]ᵢ was calculated according to the equation:

$${[ {Ca}^{2 +} ]}_{i} = {K}_{d} \times b \times \frac{R {R}_{min}}{{R}_{max} R}$$

where Rₘᵢₙ=0.61±0.02, Rₘₐₓ=1.57±0.21, b=1.09 is the ratio of fluorescent intensities during illumination at 380 nm with 0 mm Ca²⁺ and 2.5 mm Ca²⁺, and K_{d}=224 nm is the dissociation constant.

### Example 3

### Regression of Autochthonous Tumours of Breast and Colon: Biological drug delivery using multimodal nanoparticles

A multimodal nanoparticle (PEINP) of the invention which has: a supramagnetic core; highly positively charged PEI branches; and showed high efficiency of transfection of shRNAs, small oligos and miRNAs, was investigated under in vitro and in vivo conditions. PEI based nanoparticles are linked with rhodamine for the purpose of confocal imaging and visualization of these nanoparticles. The applicant further showed that a functional gene c-myc shRNA loaded on the nanoparticles were delivered to their appropriate autochthonous tumour sites, could help tumour regression effectively. The hyperthermia produced by the supramagnetic core helped in increased transfection efficacy and efficient drug delivery to the tumour necrotic core.

### Results

### Synthesis and Characterization of Multimodal Nanoparticles

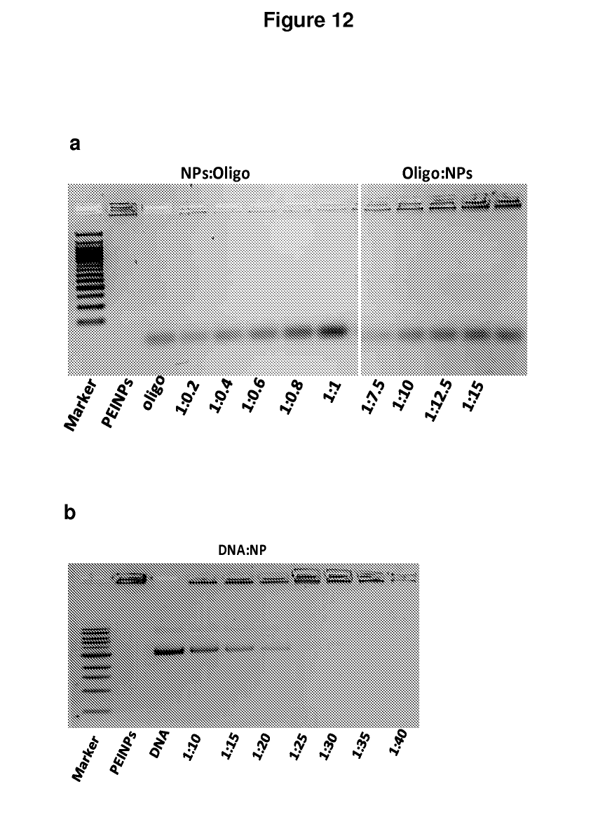

It is often essential to deliver a larger or a smaller molecule capable of producing biological drugs such as shRNA, siRNA, miRNA in the right concentration to its appropriate target(s). In such cases, the molecules are to be loaded onto appropriate drug carriers for the delivery. To assess the appropriate binding ratios of the PEINP to the vector DNA as well as to the smaller oligos, preliminary experiments were carried out with varying concentration of nanoparticles and DNA as described in the materials and methods. These experiments showed that the ratio of 1:4 (PEINPs-DNA (P:N)) was ideal for oligos to bind to nanoparticles (FIGS. 12a), and 1:20 (P:N) was optimum for binding of vector DNA to the nanoparticles (FIG. 12b), and the same has been used in all further experiments.

### Transfection by PEINPs in Adherent, Semi-Adherent and Non-Adherent Cell Lines

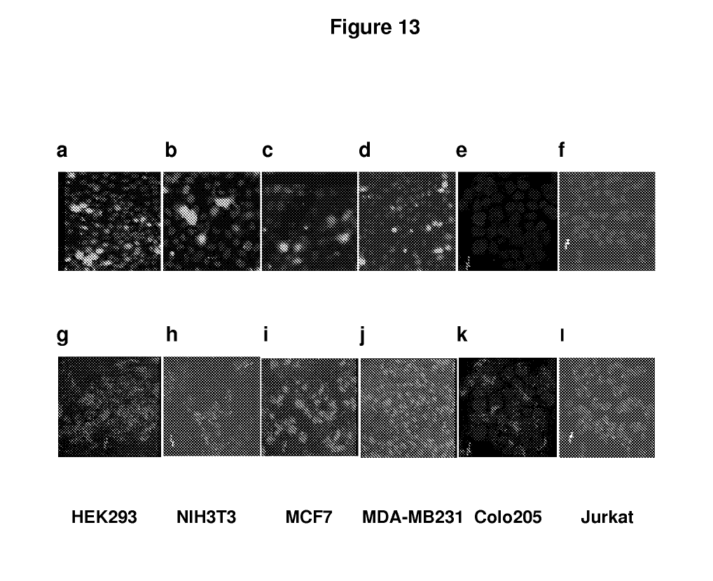

Commonly gene silencing and knockdown experiments using available transfection agents are hindered by the inherent limitation in the technology which prevents it from achieving 100% success. In this respect, a majority of the transfection reagents currently available facilitate a maximum efficiency of 60 to 70% in normal adherent cell lines like HEK 293 and NIH 3T3 (FIGS. 13a,b) and about 40 to 50% in difficult to transfect cancer cell lines like MDA-MB 231, MCF 7 (FIGS. 13c,d). The semi-adherent cell lines like colo 205 (FIG. 13e) and non-adherent cell lines like Jurkat, (FIG. 13f) still remain recalcitrant and inaccessible for using any kind of transfection reagents. Electroporation is considered the only transfection alternative, though with low efficiency, to transfect semi-adherent and non-adherent cells. As considerable percentage of cells remain untransfected by conventional transfection reagent, achieving a complete knockdown of the gene effect would appear impossible.

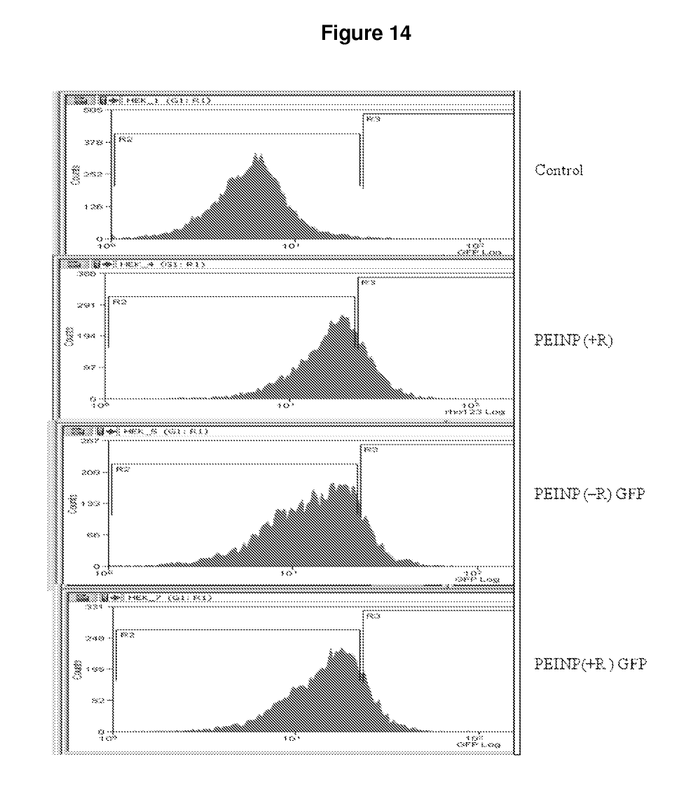

The transfection efficiency of the newly developed multimodal nanoparticles was assessed in a set of adherent, semi-adherent and non-adherent cancer cell lines independently by using the nanoparticle linked to the vector DNA which expresses Green Fluorescent Protein. The transfection efficiency of nanoparticles carrying DNA was total in all the two types of human adherent cell lines HEK 293 and NIH 3T3 (FIGS. 13g,h), breast cancer cell lines, MCF 7 and MDA-MB 231 (FIGS. 13i,j). Significant transfection efficiency in the recalcitrant cell lines including colo205 and Jurkat was also demonstrated (FIGS. 13k,l). The FACS analysis additionally confirms the efficacy of nanoparticles as transfection agent to transfect any kinds of cell lines, including, adherent, semi-adherent and non-adherent cells (FIG. 14).

Knockdown and Recovery of Target Proteins Using microRNA Oligos Linked to PEINPs

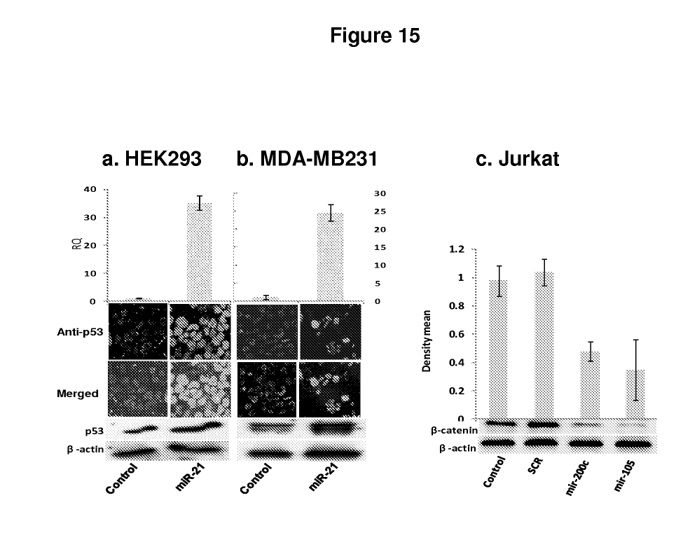

MicroRNAs are 22-23 nucleotide RNA molecules which predominantly bind to the 3′UTR of the target mRNA and shuts down the expression of the target gene either by causing a translational inhibition or complete degradation. Hsa-miR-21 is known to be a negative regulator of p53 an important tumour suppressor gene implicated in the onset of different cancer types. As a confirmation of the functionality of smaller RNAs carried by the nanoparticles, transfection studies were carried out using miR21 knockdown probes. The efficiency of recovery of the target gene was tested in HEK 293 as well as MDA-MB 231 cell lines by linking miR-21 to the linear PEINPs and assayed for the expression of p53 gene and protein using, q-PCR, immunofluorescence and western blotting. In both the cell lines efficient recovery of p53 gene was noticed (FIGS. 15a,b). Similarly Jurkat cell lines were transfected with miR 200C and miR 105 mimics after linking to PEINPs and were assayed for the knockdown of target protein b-catenin by q-PCR and western blotting (FIG. 15c). Complete shutdown of target proteins in these non-adherent cell lines like Jurkat showed the efficiency of PEINPS as a novel transfection agent to be used in vivo.

### A Functional Gene is Silenced by PEINP Mediated Transfection

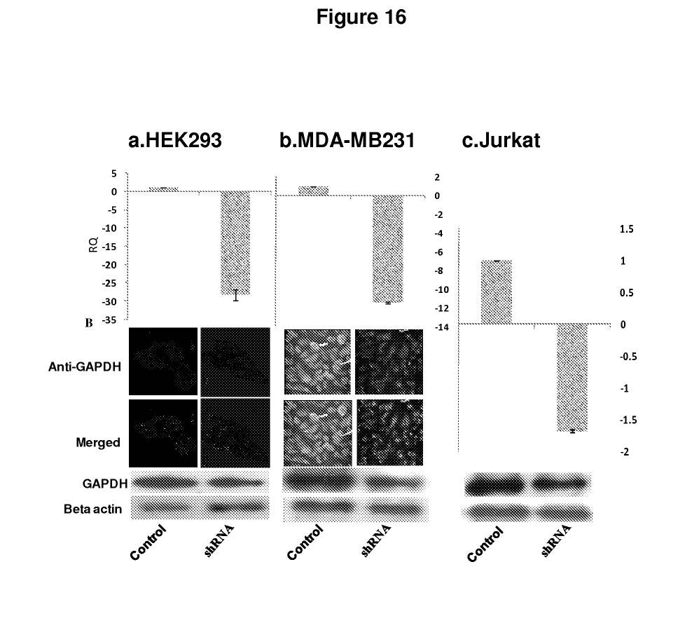

To reaffirm the higher efficiency of transfection of large vector DNA construct by the PEINPs, a positive control gene GAPDH shRNA cloned in pGIPZ vector that would lead to formation of shRNA against the target gene GAPDH and thereby lead to its shutdown was linked to the nanoparticle in 1:20 ratio (P:N) and used for transfection. The efficiency of GAPDH shutdown in 3 different cell lines like HEK 293, MDA-MB-231 and Jurkat cell lines was confirmed using immunocytochemistry, q-PCR analysis and immunoblotting. (FIGS. 16a,b,c). These GAPDH shutdown experiments have provided evidence for the ability of the PIENP to carry large DNA vectors, transfect different difficult to transfect cell types, deliver the DNA in the cells which in turn shuts down the target gene efficiently.

Functional Shutdown of Oncogene c-myc Using PEINP In Vitro

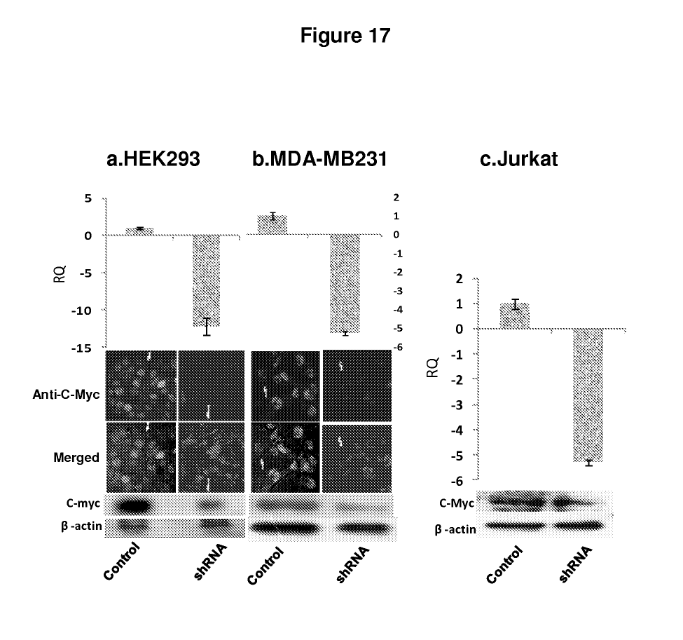

C-myc is a central oncogenic switch between oncogenes and tumour suppressor genes. The genetic alterations of which accounts for one seventh of US cancer deaths and as such, most research is directed towards understanding the role of c-myc in cancer biology with the hope of novel therapeutic approaches. C-myc shRNA cloned in an appropriate vector pGIPZ (Open biosystems) was selected to show a functional gene shutdown in vitro. C-myc shRNA linked to nanoparticles (1:20) were transfected in all the 3 cell lines as described in materials and methods. Immunocytochemistry experiments showed shutdown of target gene c-myc by c-myc shRNA several times compared to the control. This was further confirmed by q-per and western in all the cell lines (FIGS. 17a,b,c). Thus PEINP provides a useful carrier of biological drugs in different cell lines.

Biodistribution and Penetration c-myc-shRNA-PEINPs in Breast Cancer Models and Regression of Autochthonous Tumours

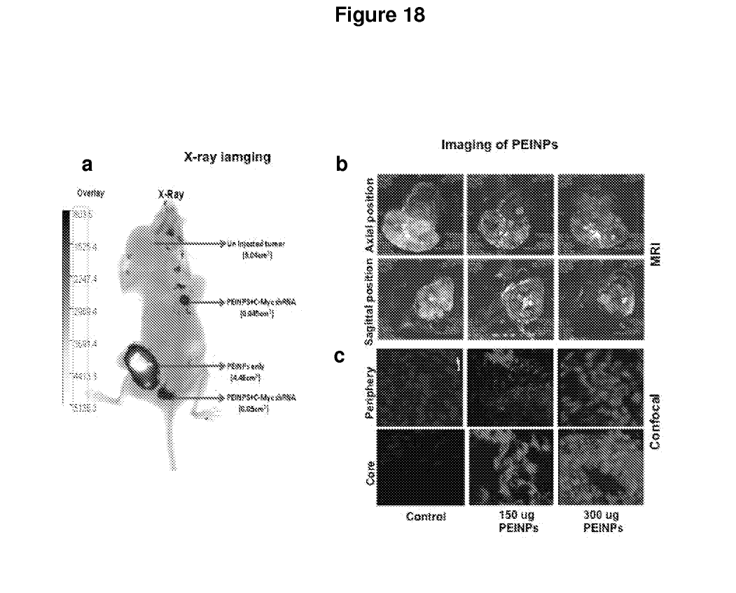

In order to understand the biodistribution of PEINP in vivo, Brca2/p53 knockout murine breast cancer models, which develop natural tumours between 6 to 8 months of age, were selected. Intratumoral injections to the periphery of the tumour were carried out using c-myc shRNA bound to the PEINP (1:20) in 6 cohorts each of 3 to 4 mice, C-myc DNA, PEINPs alone and DNA bound with arrestin were used as controls in the similar replicates to test the penetrability and distribution of the nanoparticle to the core of the tumour. The injected mice were kept under a magnetic field to allow for penetration and localization. The live imaging showed accumulation of Rhodamine restricted to the tumour alone with a maximum intensity at the core region (FIG. 18a). Though injection was done at the tumour periphery; equal distribution of PIENP was noticed throughout the tumour core and periphery which were also viewed by confocal microscopy (FIG. 18c). The iron core of the PEINP traced the NP distribution to the necrotic core of the tumour by nuclear magnetic imaging (FIG. 18b). The c-myc-shRNA-PEINP treated tumours stopped growing and also started showing the signs of regression compared to controls (FIGS. 18d,e), Survivability of the treated mice cohorts were prolonged by 20 days compared to the untreated cohorts (FIG. 18f), q-PCP and Immunohistochemistry of the treated tumours showed significant reduction in the c-myc level compared to untreated tumours (FIGS. 18g,h). Biodistribution of PEINPS was restricted to tumours alone and not to other organs indicating targeted delivery of PEINPs to the necrotic core of tumours.

PEINPs-c-myc shRNAs were Preferentially Retained in the Colon

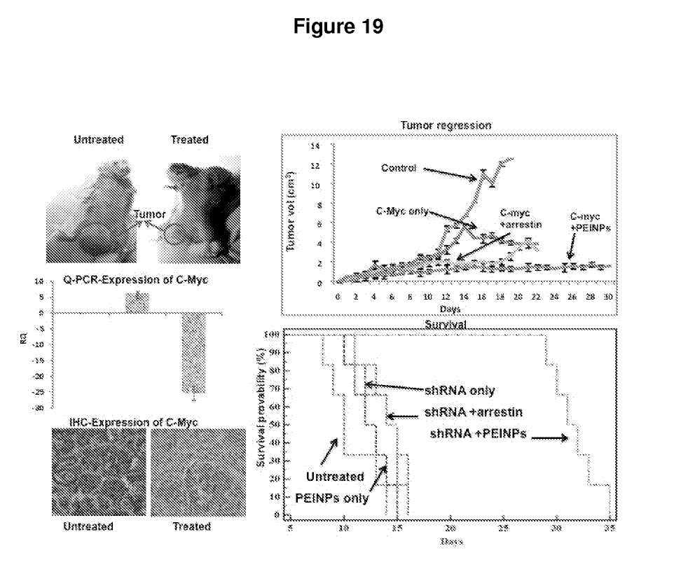

Colon cancer models (Apc fl/fl) induced by tamoxifen and b-naphthoflavone injection brings about severe lesions in the gut and the mice usually die within a span of 7 days. C-myc-shRNA with turbo-FP as reporter gene was orally delivered using nanoparticles to these models. The confocal imaging of the mice that were orally administered with PEINP showed the presence of rhodamine predominantly in the colon indicating the retention of the PIENPs in colon region (FIG. 19a). Only trace amounts of rhodanine was observed in the other organs like oesophagous, stomach, small intestine and large intestine (FIG. 19a). Turbo-FP DNA also was detected only the gut which indicated the preferential retention of nanoparticles in the gut with the release of DNA (FIG. 19b). No turbo-FP DNA was seen in the gut region when naked DNA or DNA along with arrestin were orally administered which indicated that naked DNA would not be retained in the digestive tract This clearly proved that PEINPs could be used as ideal carriers to deliver biological drugs preferentially to the colon.

### Embryofection of PEINPs-DNAs in Mice Embryos

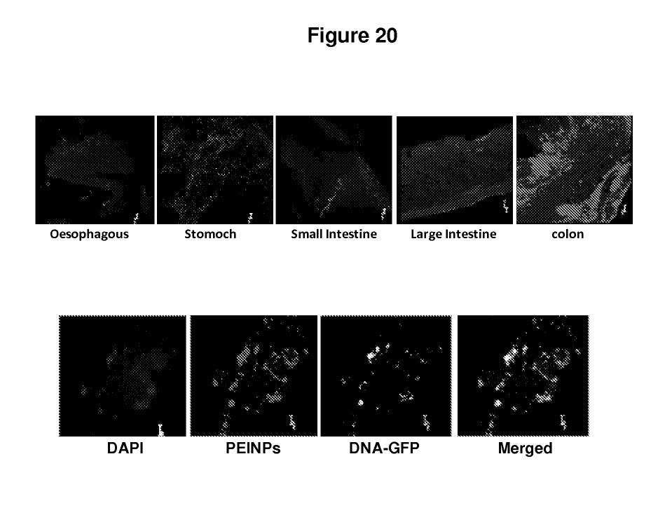

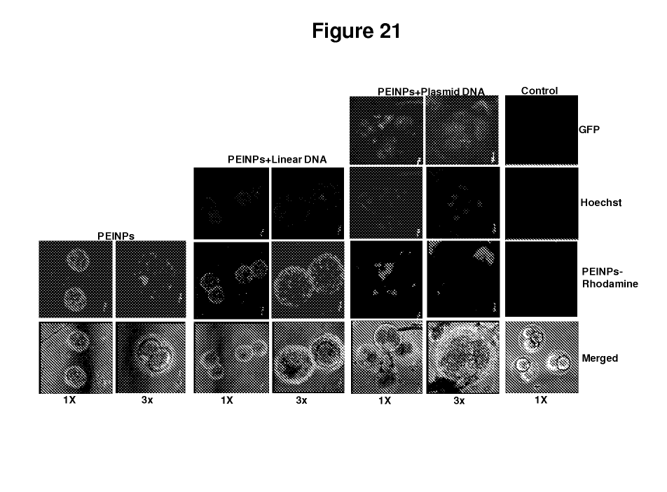

The transgenic and targeting technologies are limited by the low rate of transgenic production in higher animals. Mouse is an ideal model system that reflects various human disease conditions in vivo. These models systems form an ideal platform for monitoring the initiation and progression of various diseases including cancer. However, the rate of production of transgenic mouse is limited by its technology to only 4 to 6%. This is basically due to the poor survival rate of embryos undergoing the trauma due to microinjection. To explore the possibility of obtaining better frequencies of production of transgenics by minimizing the manual handling, it was investigated whether it was possible to introduce the genes using PIENP. The DNA was stained with Hoechst stain, washed and then used to link to nanoparticles for visualization of introduced DNA inside the embryos. The embryos were incubated with PIENP-DNA complex and were retained on magnetic platform. After 48 hrs GFP expression were also visible inside the embryos (FIG. 20). This indicated that nanoparticles had managed to deliver the DNA inside the embryos during incubation. This was termed embryofection for this PEINP mediated DNA delivery in embryos. These embryos need to be transferred to surrogate mothers to establish the production of transgenic mice using this technology (FIG. 21).

### CONCLUSION

The present invention provides a pH sensitive, polyethyleleimine based multimodal nanoparticle capable of delivering biological molecules to their appropriate destination. To determine if these particles could be used as drug carriers, it was tested whether PEINP could be used for delivering shRNAs, small oligos and miRNAs to both adherent and non-adherent cells with the efficiency equal to that of viral transduction. It was demonstrated that the PEINP could be used as carriers of shRNAs or miRNAs for targeted silencing of functional gene, for example, in the down regulation of c-myc gene that led to the regression of cancer in mice. Thus, the PEINP of the invention may be used in non-toxic as well as non-invasive therapeutic approaches for combating cancer and other diseases and disorders, and for the validation of innumerable drug targets.

1. A nanoparticle comprising a polymeric nanosphere and one or more detection agents, said one or more detection agents for use in detecting the location of the nanoparticle, wherein the polymeric nanosphere comprises poly(glycidyl methacrylate) (PGMA).

2-3. (canceled)

4. The nanoparticle according to claim 1, wherein the one or more detection agent comprises one or more surface modifying agents.

5. The nanoparticle according to claim 4, wherein the surface modifying agent is a polycation.

6. The nanoparticle according to claim 5, wherein the polycation is selected from poly(ketimines), poly(amino acids), poly(guanidinium), poly(alkylamines), poly(arylamines), poly(alkenylamines), poly(alkynylamines), poly(imidazoles), poly(pyridines), poly(pyrimidines), poly(pyrazoles), poly(lysine), branched or linear poly(ethyleneimine), poly(histidine), poly(ornithine), poly(arginine), poly(asparginine), poly(glutamine), poly(tryptophan), poly(vinylpyridine), copolymers thereof, and cationic guar gum.

7. The nanoparticle according to claim 4, wherein the surface modifying agent is polyethylenimine (PEI).

8. The nanoparticle according to claim 4, wherein the surface modifying agent is one or both of PEG and PEG copolymers.

9. The nanoparticle according to claim 1, wherein the detection agent comprises one or more imaging labels which can be detected using fluorescence and magnetic resonance imaging (MRI); fluorescence and positron emission tomography (PET); fluorescence and x-ray computed tomography (CT); or fluorescence and MRI and PET and CT.

10. The nanoparticle according to claim 9, wherein the imaging label is iron oxide nanoparticles.

11. The nanoparticle according to claim 9, wherein the imaging label is Rhodamine B (RhB).

12. The nanoparticle according to claim 1, further comprising one or more therapeutic agents selected from DNA, RNA, polypeptide, antibody, antigen, carbohydrate, protein, peptide, enzyme, amino acid, hormone, steroid, vitamin, drug, virus, polysaccharides, lipids, lipopolysaccharides, glycoproteins, lipoproteins, nucleoproteins, oligonucleotides, immunoglobulins, albumin, haemoglobin, coagulation factors, peptide and protein hormones, non-peptide hormones, interleukins, interferons, cytokines, peptides comprising a tumour-specific epitope, cells, cell-surface molecules, small organic molecules, small organometallic molecules, nucleic acids, oligonucleotides, metabolites thereof, and antibodies to any of the above substances.

13. The nanoparticle according to claim 12, wherein the therapeutic agent is a drug.

14. The nanoparticle according to claim 12, wherein the drug is a slow release drug.

15. (canceled)

16. A transfection agent for transfecting a cell with nucleic acid, comprising a nanoparticle according to claim 12.

17. The transfection agent according to claim 16, wherein the therapeutic agent is one or more selected from the group consisting of plasmid DNA, vector DNA, siRNA, shRNA, genomic DNA, small or synthetic oligonucleotides nucleic acids and oligonucleotides comprise genes, viral RNA and DNA, bacterial DNA, fungal DNA, mammalian DNA, cDNA, mRNA, miRNA, miRNA mimics, miRNA inhibitors, piRNA, RNA and DNA fragments, modified oligonucleotides, single stranded and double-stranded nucleic acids, natural nucleic acids, and synthetic nucleic acids.

18. A transfection agent according to claim 17, wherein the ratio of the nanoparticle to nucleic acid is approximately 1:20.

19. A transfection agent according to claim 17, wherein the ratio of the nanoparticle to nucleic acid is approximately 1:4.

20. A method for modulating phenotypic changes in a cell, tissue or a subject, the method comprising introducing to the cell, tissue, or subject a transfection agent comprising a nanoparticle according to claim 12.

21. A method according to claim 20, wherein the transfection agent comprises a miRNA mimic to increase the function of endogenous miRNA to assist detection of a phenotypic change in the cell, tissue or a subject; or a miRNA inhibitor to decrease or eliminate the function of endogenous miRNA, increase expression of a target gene, and thereby modulate a change to a phenotype in the cell, tissue or subject.

22-26. (canceled)

27. A method for treating a disease associated with undesirable levels of a polypeptide in the cells, or tissue of a subject, the method comprising administering a transfection agent comprising a nanoparticle according to claim 1 to the subject.

28. A method of modulating gene expression in the central nervous system of a subject, comprising administering to the subject a transfection agent comprising a nanoparticle according to claim 1.

29. A method for transfecting a cell comprising introducing to the cell a transfection agent comprising a nanoparticle according to claim 1.

30. A method for modulating the expression of a gene in a cell, tissue or a subject, the method comprising the step of introducing to the cell, tissue or subject a transfection agent comprising a nanoparticle according to claim 1.

31. A method according to claim 30, wherein modulating the expression of the gene increases, decreases, or eliminates levels of polypeptide product of the gene in the cell, tissue, or a subject, as compared to normal levels of the polypeptide within the cell, tissue, or subject.

32. A method according to claim 30, wherein the transfection agent comprises shRNA or siRNA complementary to the gene, and expression of polypeptide product of the gene is decreased or eliminated.

33-35. (canceled)
